SEQUENCE LISTING (19 sequences)

SEQ ID NO: 1 - DNA, 7514 bp, Homo sapiens
cgccatggcggcgccgggttgcggaccctgagcgccggcgcggggcgcgcaccatgaactcgtgggacgcgggcctggcggggctactggtgggcacgatgggcgtctcgctgctgtccaacgcgctggtgctgctctgcctgctgcacagcgcggacatccgccgccaggcgccggcgctcttcaccctgaacctcacgtgcgggaacctgctgtgcaccgtggtcaacatgccgctcacgctggccggcgtcgtggcgcagcggcagccggcgggcgaccgcctgtgccgcctggctgccttcctcgacaccttcctggctgccaactccatgctcagcatggccgcgctcagcatcgaccgctgggtggccgtggtcttcccgctgagctaccgggccaagatgcgcctccgcgacgcggcgctcatggtggcctacacgtggctgcacgcgctcaccttcccagccgccgcgctcgccctgtcctggctcggcttccaccagctgtacgcctcgtgcacgctgtgcagccggcggccagacgagcgcctgcgcttcgccgtcttcactggcgccttccacgctctcagcttcctgctctccttcgtcgtgctctgctgcacgtacctcaaggtgctcaaggtggcccgcttccattgcaagcgcatcgacgtgatcaccatgcagacgctggtgctgctggtggacctgcaccccagtgtgcgggaacgctgtctggaggagcagaagcggaggcgacagcgagccaccaagaagatcagcaccttcatagggaccttccttgtgtgcttcgcgccctatgtgatcaccaggctagtggagctcttctccacggtgcccatcggctcccactggggggtgctgtccaagtgcttggcgtacagcaaggccgcatccgacccctttgtgtactccttactgcgacaccagtaccgcaaaagctgcaaggagattctgaacaggctcctgcacagacgctccatccactcctctggcctcacaggcgactctcacagccagaacattctgccggtgtctgagtgaaggaccgcgctcctgctgaagagtttagaatgaggcagcggtgagaagaagggtgggagggcgtgggggcccctgggtggacaccaccagccaccagtccctggcatgcccagtgatcctggttccctggcttgtaggggctccagagcctgcttcctggttcctcaagggcagatattggacactccttatttgtcaccaaaggatgactgtaggccgtgtgctggcctttctttctaagaagctgctttgagctcctggactcacctgaggctccctgggggatgacactcagttctgtcactgtcaaggatgcagagagctggtggtaggtgggaagcatggtgtccacctgcctgctgaccactggacgctgctccatgctgaagaaaagtgacagtctccaggggacatttcagccatgctgaaagggaggctggcagtggtcatttggcccggatctaacatggcacctcgtctccacagggtagtggtggctgcttcaacccaaatattattcagctggtactaacgacattgtgcccagctgggactcttgggctctgtgcctgagggaaaatgtttcacaactagtggctgcccaattgctgctgaccagttgtcttagaaatggtcaattggattcaactttagtcctctccttccccctaaaagcgaatgtttgtgtgtgcagacaatcttagcatgaaaatggtttaaataggctggtcctacatgtattaggttctttcaagtttgactgggaggtcacctttttctgatttacaagtcctaattgttggagctcagtaaaggtaggaggaaggtggctggttggtcctcccttccccctgtttgtgacctgaatttacaggaagtgtttcaacttgtcttatgcatcttatctggcatgtcctgggagatggatgggcaagaactggcctgagcagggatttttgccttgattttaagtcactgggttccattgtcctggcacctccatttccttagtttctgtaagcctgttaacagaaagtagaggctattcaaggttatcaagaaagtgccctgtgctaatgatgagacagtgaattttttttttttttcgagatgggagtttcactcttattgcccaggctagagtgcaatggtgcgatctaggctcaccacaacctctgccttccaggttcaagtgattagagacagtgaaatttttatggaatactgcacctataaaacacatgccaatctgttagcagtcctgacttgatttaataaaaaccaaggagagccacccctgctttctggccatctcttctttgtctaccatttcaaattgacttgtgaatactcagagcagttatttttcaatcctccactctaagtgattcctccagggtgggaagcacggggagcaatggaatcagatggctcattctctccttccagctctttacatcctaactttggaggtatagaaggaaggatgtggtccaacgggaaggaagtgaagagtctattggtttttacttctaggcagtcccaatttttaaaggcagagaagtatctgcatctttatttgaaaagacgtcttattaactcttagctcctcagttagaaaacaattcacctggcttagtcttgtctagtttcaaagttaccatggagactagaatggagacaggtgtagcttccaaggaggccttggcatcttgcagttgctgtcagagatgggctcatatctgcttggggaagccaatggggcacaatccttcccaatttcatgatctggggcatcatgtcggtagctgaagtcagccacggtgggagtgtttataccatgaaaatcagcaagtgctgtaaatcaggccttttgccccacagagagctgattgtgaaacatctactagcacaccactgccagacatgactgtgtgtgtccctgaaacaaaattgctctggctcagagctcctttggccactcatattacaatagcaacagacatttgcttcctggtggcaagtaactgtgcaccctccaacatcccaaccccagaggaaccagtgggattgaaaaccagcagaagccaagacaggatgcacagctgaattggaggagagtagctcccttctcacacaaacgctttcaaagttgggcaaaagtggcccagtgcgtgaggtcagtcaacagccaaattccagcagcaaaatacattttcatcaatatcactttgggagaataactgtggcacatgctctttctgctttctctgtactgtataggcaacatgttgtactcccagtagcaactagatctcttttacctcttctttagccaagaagcaagatctgtcacccacaacaccacatatcttgaaaagatgcttttcataattttcactatacctctaaaaccaattgtaccgacatcacgtagacaaaaactccagataaatccattgattgggtattttcattattatttggacacatgaattaaatcccaagggaaagtgagaagaaaattaatgtattagcaaccagtgttagccaactctgctgtaattacctttagataaatgtttattttcatctctatacagtctatcaaatttgccccctggctttattactcccaattgaattatctacctcattttgttaatttaatttatcaaaggtaacattgttttctcaggtataacacatagaaacaccaagattttcagcatctgaatcaaactaaaattacttgcttgacttcactccagggtgagacttgacctttaaggcccaaactctggtgaactgttggactgggtagaaatctttcaagaagcttgaaaataaggccataaaggaggggctttcaaattcctaaatccacatatagggctgagttcctagtcctacacaatcagaatttgcctttttaagtcacgctgcaactattccggctttttatatggcacctttctgagggaggggattttacagccgagggacctccaaagggaacgtccatttgcatgtgtgccctggtatttagggtttgtctcatggtcagctcgcctctcaaagatggctcaccaatcacctgcctgtttttacttagaaagcaggcttgggcaggaggggaaatggatttgaaccatctcagaccaactctatctcttctagcactcagctcagcctcctgtccccatgagcaataaaactctctccttatgtctgtgaaattcgttttctttttcatggtgcctttctttctaagaaaagatagaaagaatggggccaggttcatgtcccagttgtcacccttttacatgcttgggtgcaggtagatgaggccttcccatggctacagtgtgacccttggaggaaccaaaggctgtgcattgttagaatttagcccagatgcaggataaactagcttgcctgagtttaatgaggatattatatcaggtgccaggaatcccttgggtcagctcctatcctctggacggggagcatcatgggaaaaagaggtccgagtcatatttagggcttcatgtgtctggaagggagccattagctcttaaattattcacagaagtcaccacggaaggttgtttaagatgcagattccagcatcccctcctcagagatcaatttcagcagggccaggcaggacctgggtttccgcacttttaatcatcgatgcccaggtaactgataggagttttcttcaaaccatcccctaaggagaaatgatcttagacacttggataaactctatagaaaattgagtgtttctattaataactatctaggtttcaattgtcttgtatctttccctaattctccaaacagttcaatctccagctttattcaatggctgggccattttgttcttggagggacaaaattgacctctccatgaaattgcaaaaacccatcttcattcctttttatggggctccaggtcataaggatgagatatatttggggtactggcagaggatggcagagctggctggctctgagccaggaagccttgttagaggaaggaactctggcattcagaggtgtccccttgggggcacttgcttgggtcttggatcaaactctactggggcagcctggctgtgggaggtgatgacatctgccacctctgccactacccagagcttgtcctgccctcttcccagccagcctcacacacaggagctgagccaggctgaggcaaggtcagcgagagacagttctccatgccagtatctgggtgtgggtctcttgttggctcccagctcatcccgtgtggctgacccttccaggtcacttggagcagccagtaatgacagtgtcctaacagctggtgggtatggaggaaagactagacatcaggctatggactcacatggagggccctggtgttggctctgggacctgggctgggccggacctggggagagggtgggtggagcttggcctctgcagtttgcgcttgcaggttcaggggagaaagttgtttgagatcagggtttaggggtagtgtttcccattaatcttcatcataatcttttgatgtaggtgctgttattatctctacgttacacatctgggaggatttgaaagtcctggagacagggaggttttggggagaatgcaaaacagggcagggctctcaaggagggtggaccagtgaccatggggaaactgagtgtttctattaataagtatctaagtttcaattgtcttgtatctttccctaattctccaaacggttcaatctccagttttattcagggactgggccattttgttctcagagggacaaaactgatctctccatgaaatcccctgcacctcactctgggcagctctaggctcagcagatgtttttctgccattaggaaacagccccttactctcagctcctgtgctaacacccatacccaccagctgggcgttgggacctctcccgtcatatggctgccacttgcttgggaaggaattctccagactccttttactttgtatgtgtttctaggggaattctgagggaagaagctttgcagcaacctctgtgcccagaacatacggctcacaagtcctttgtgccgacagggaatctatctaattgttttagtgtgtgaggctcattgtcagcctagatttccctgaaactctgggccagcagcccccaggagcatcagtaggacccagcatttcccctggcaccaaagtggacctctttgcaccttgcagccgggagaactgacttagagggccctggtgttggctctggaacctgggctgagatggattcggggagaggctgggtggagcttggcctctacggtctgttcttgcagattcaggagagaaagtggtatgagagcagtgtggtttgtaagttccccaacttccccgctggctcacactgtctccccagaccaatggcctattagcccccaaaaagttagtccaaccccaggccagctgcctttgctctgctgtggcctaagttctggcctggtcaaaggaaggtggcaggaactccaggcttgctcctgcctcctcacacctcattccacaaatgcggagtgaaagagcaggagccagacacatttgggggttgactgtctagaaaggaggaatcagaggtcccaaggaacacaatcgtgcaacttcctagggaatctgcatcaggagaaagaagcggctaggacacacagtggcttcgctcagcctggccacgacatcactgtgcagaagaagagatctgtcaggggtccagaggagcggagtcctggagcctctgggagggtggcatgtggcagctgcaggctgtggtccagaactatgagcagacttgcctgtcactctccaaacatttactgaacacctactgtgtgccaggccctgtgctttgtcaggctctgggggagacaggtcattactctgcagtgtgggaagcggggctgccctggaagtgtgtactcagtgctgtgggggtacagagggagggctggagaacttagctggtgggaaaggagggttctggaaggcttctcagaggtagtggcattgggccagaacttacagtagggaagagcatgtggcaaggcacagaggagatgggagcctgcctggtgaggggtccagtggtgacagagctcagtgtgggtccagagatggtgtcaggtgagggctgagaggggagggcaagtgccccttgagatgggctgaacgtgatgatgatctgtggccagtgggagatgatagaagtcttgaggttgggaatataagaattaggtctcttttagaaagagaggacatggagaaaatccatatatttttagctaccaaaaaagagaaggaagaaagaaaagaaataagaagagaagcaagacagaaaagaaggaagagagaaggaagaatggcaaaggaaggaagaaggag

SEQ ID NO: 2 - DNA, 1014 bp, Homo sapiens
atgaactcgtgggacgcgggcctggcggggctactggtgggcacgatgggcgtctcgctgctgtccaacgcgctggtgctgctctgcctgctgcacagcgcggacatccgccgccaggcgccggcgctcttcaccctgaacctcacgtgcgggaacctgctgtgcaccgtggtcaacatgccgctcacgctggccggcgtcgtggcgcagcggcagccggcgggcgaccgcctgtgccgcctggctgccttcctcgacaccttcctggctgccaactccatgctcagcatggccgcgctcagcatcgaccgctgggtggccgtggtcttcccgctgagctaccgggccaagatgcgcctccgcgacgcggcgctcatggtggcctacacgtggctgcacgcgctcaccttcccagccgccgcgctcgccctgtcctggctcggcttccaccagctgtacgcctcgtgcacgctgtgcagccggcggccagacgagcgcctgcgcttcgccgtcttcactggcgccttccacgctctcagcttcctgctctccttcgtcgtgctctgctgcacgtacctcaaggtgctcaaggtggcccgcttccattgcaagcgcatcgacgtgatcaccatgcagacgctggtgctgctggtggacctgcaccccagtgtgcgggaacgctgtctggaggagcagaagcggaggcgacagcgagccaccaagaagatcagcaccttcatagggaccttccttgtgtgcttcgcgccctatgtgatcaccaggctagtggagctcttctccacggtgcccatcggctcccactggggggtgctgtccaagtgcttggcgtacagcaaggccgcatccgacccctttgtgtactccttactgcgacaccagtaccgcaaaagctgcaaggagattctgaacaggctcctgcacagacgctccatccactcctctggcctcacaggcgactctcacagccagaacattctgccggtgtctgagtga

SEQ ID NO: 3 - protein, 337 aa, Homo sapiens
MNSWDAGLAGLLVGTMGVSLLSNALVLLCLLHSADIRRQAPALFTLNLTCGNLLCTVVNMPLTLAGVVAQRQPAGDRLCRLAAFLDTFLAANSMLSMAALSIDRWVAVVFPLSYRAKMRLRDAALMVAYTWLHALTFPAAALALSWLGFHQLYASCTLCSRRPDERLRFAVFTGAFHALSFLLSFVVLCCTYLKVLKVARFHCKRIDVITMQTLVLLVDLHPSVRERCLEEQKRRRQRATKKISTFIGTFLVCFAPYVITRLVELFSTVPIGSHWGVLSKCLAYSKAASDPFVYSLLRHQYRKSCKEILNRLLHRRSIHSSGLTGDSHSQNILPVSE

SEQ ID NO: 4 - DNA, 1014 bp, Mus musculus
atgaactcgtgggacgcgggcctggcggggctgctggtgggcactatcggcgtgtcgctgctgtccaacgggctggtgctgctctgcctcctgcacagcgctgacatccgccgccaggcgccggcgctcttcactctcaacctcacgtgtggcaacctgctgtgtaccgtggtcaacatgccactaacactggccggcgtcgtggcacaacggcagccggccggggaccgcctgtgccgcctggccgccttcctcgacacctttctggccgccaactccatgctcagcatggccgcgctcagcatcgaccgctgggtggctgtggtctttccgctgagctaccgtgccaagatgcgcctccgagatgccgccttcatggtggcctacacgtggctgcacgcgctcaccttcccggccaccgcgctcgccctgtcctggctcggcttccaccagctatatgcctcgtgcacactgtgcagccggcggccggacgagcgcctgcgctttgctgtcttcaccagcgccttccatgcgctcagcttcctgctctccttcatcgtgctctgcttcacgtacctcaaggtgctcaaggtggcccgcttccactgcaagcgcatcgacgtgatcaccatgcagacgcttgtgctgttggtggacatacaccccagtgtgagggaacggtgtctggaggaacagaagcggaggcgacagcgtgccaccaagaagatcagcaccttcatagggaccttccttgtgtgctttgcaccctatgtgattaccaggctggtggaactcttctccacagcacccattggctctcactggggagtgctgtccaagtgcttggcctacagcaaggccgcttctgaccccttcgtgtattccttgctgcgacaccaataccgcaggagctgcaaggagctcctgaacaggatcttcaacagacgctcccttcactctgtgggcctcacaggtgactctcacagccagaacattctgccagtgtcggaatga

SEQ ID NO: 5 - protein, 337 aa, Mus musculus
MNSWDAGLAGLLVGTIGVSLLSNGLVLLCLLHSADIRRQAPALFTLNLTCGNLLCTVVNMPLTLAGVVAQRQPAGDRLCRLAAFLDTFLAANSMLSMAALSIDRWVAVVFPLSYRAKMRLRDAAFMVAYTWLHALTFPATALALSWLGFHQLYASCTLCSRRPDERLRFAVFTSAFHALSFLLSFIVLCFTYLKVLKVARFHCKRIDVITMQTLVLLVDIHPSVRERCLEEQKRRRQRATKKISTFIGTFLVCFAPYVITRLVELFSTAPIGSHWGVLSKCLAYSKAASDPFVYSLLRHQYRRSCKELLNRIFNRRSLHSVGLTGDSHSQNILPVSE

SEQ ID NO: 6 - DNA, 6731 bp, Mus musculus
tggtgcttagcatctgtcaaggtgtagaagagaaaggccattgttctctctgacttgcaggtgctttagattaaccccctccatccttgaatctatgtgagggaagataattagtgtgatgggggagacacaacagctcccttagggaaactgtctatatttggtttctttcagactgaggtctggctatgtacagtcatcactggccatctcaccaggctctgaaatacagtgtaggtggtcactggaggaggtagcatcccatagaaaggcaagagactgaggaagctctgaaagttgtcacctgtctgtccaccaagcacctttgaaactacggagtctggactagggatagaagagacacagttcccacatgtttacctgaggggaggctgagaccctttatgtcccaaactctgatgatctggatcaaacagagacatccgctatcctcccattctctgaacagttcatccacatgcctaataagcttatactcgctggctcgccacacccagaatggacaggacagcagctggcccaggacacaatgagtgtccagaaccagctacaccaggactacttctgatgaccatggacagagtgtgttcttgactgatggcatgcccaagggatgtcccggtgagcctcaagtgaccttgcaccatatctgagagtgtagctgaatgactagagagtgaagagacgaagctctggagactcagtgtccagcagtacagcaactgcctgcagatagatgaggggagatctcaggagatctctggccataatgcaagacagaactgtgaattctatcccagctcagcagaagcctagccaagctccccagttcatcttagataacagcagcaatcctagatgctgggcagctatggggcaggtggactgggtcaaggtctggtcacctaggcagggacaagcagcactgggacatgagggtgcccagacattcttcccttttcctagatttcttgactcagcctgtctccagctcatataggggaggatagaaaggaaggggaggaggggagccactgggagccagccattctgcaagggagccggtagggctccttccctccaagtgtgtgtgtgtgtgtatgtgtgtgtgtgtgtgtgtgtgtgtgagagagagagagagagagagagagagagagagagagagaatcggtgggatttccttctcagaagcagccagtttcctaagagaaaaaagagagaaaggagaaagaaagggaggagaaaattggaggtgggagagacagaagggggagggggcagaaagtgagagcacacaggagactgagcacagaggcagagagagggagacagagagaaagccacaaagcagaagggtgtgcggagcagagccagagctgagagggacccagtaaggcgggtgcaggcagacgcaaagccagtgaccacaggagactgcgaccccggattccagggcacttcaggaggtcagaggagcacaggccaagaggtggtgcgggactgggagtgaacactcgcctaggtgatcgccggtgaggtgaccccagactccgagtggttccgtgcccaggtgctcgcctgggaggtggcgcggggctccgggaggctgcgtgcctaggtgctggcccagggggtggcgcgggcttctgggttggtggatgccctctgggtagccatggcgacaagggccaggggtcgctgggctgccgtgtcgcctaggggctgagtcgacagcaggctgcgccatggcggcgcggggttgcgggccctgagcgccagcggcgggcgcgcaccatgaactcgtgggacgcgggcctggcggggctgctggtgggcactatcggcgtgtcgctgctgtccaacgggctggtgctgctctgcctcctgcacagcgctgacatccgccgccaggcgccggcgctcttcactctcaacctcacgtgtggcaacctgctgtgtaccgtggtcaacatgccactaacactggccggcgtcgtggcacaacggcagccggccggggaccgcctgtgccgcctggccgccttcctcgacacctttctggccgccaactccatgctcagcatggccgcgctcagcatcgaccgctgggtggctgtggtctttccgctgagctaccgtgccaagatgcgcctccgagatgccgccttcatggtggcctacacgtggctgcacgcgctcaccttcccggccaccgcgctcgccctgtcctggctcggcttccaccagctatatgcctcgtgcacactgtgcagccggcggccggacgagcgcctgcgctttgctgtcttcaccagcgccttccatgcgctcagcttcctgctctccttcatcgtgctctgcttcacgtacctcaaggtgctcaaggtggcccgcttccactgcaagcgcatcgacgtgatcaccatgcagacgcttgtgctgttggtggacatacaccccaggtgagtgccctgacccaagactctgagcacttggtattgagttaggaaggtcccagcatcccgggagatcccctttctgcatcaatggcattaagcacgggtggaatagcagatcctgtttaaacgggtgcctagagcttcactttctgaaacatgggactgaaaccccagccagaacatcttgttacccttatactctgggcagtgaactctgttcacttttcgaagcttgaaagtggtttggatctgagaggggtatctagaggattggaaaacccttaagaactcttcaaatggtgggtgttattcctctgggtctttgcagtcctgggtgtggtgccacctggatctcctcgcatgagtcctcattaaacagaatccatagcaccttctcccagtgatgagctctgagggccccagttcttcttgccttcctagaaggttcagtccatcgttttctagacttggaacctcaaggtcccttaaagaatttgtatctcaggtttctgaaaccaagcctgcccaccacttccattctgttgacttgctatttctttggagaagaaggagggaggcaaatggaaagatggactgggaacctccgtggtccttagtgttccttagggagaagaggaggaaactgtcaatcgggctgttctgagctcacctcatctttcctcttgagactaggacatcaagagggagggtgcttctgtggcctctgccagtttatatacattaaaagcagactccctctctgctgcctgggacttaggagtccacttgacacagaagatacagcacacagggagcaatagtgatggacagttaggcttcaatgtgtgagcagagccccaggatgtggggagccttggggaatggaatgtagaccccaatgtatattcaccctccagggcacagtggacagactgagcaaaagcctttgtgaagcttccctgccttgagcaatgtcactatgctgaatgttactctacccgctcatcagaagggcatggctgagtgtgtctactgccaccccctgagcacacattcaagatgacatggtggtccagtctttcccaaataaaatcttccccacagaagcttaaaccaaatgtgcttacatcagctctctcagcaatggtctgtgcagtttagtcatttcaggagagagtctgaagccacacttgtagaaacattcccgttcattgtccttcactttagaaggggctggggctgggtccgggtccggttctggttctggttctggggctagttctgggtttgggactggaaccgggactggggctactgctggggctggggctgggtttgaggctagaactgaggctggggctggggctggggctggggttggggctggggctggggctggggctggggctggggctggggctggggctggggctggggctggggctggggctggggctggggcagtttgtgaggatgcatggatgtgttagcgcttgcagacttgaacaagtcctagaaagccccctcatgtctagcctcccaatggagggaaagaaaggctgagccaggagaggagcccattgtatttagagcaatgcaaagtccaccaccagctctcagcccaccagagaggagggtggaggccactttgaattctatttagagacagggacttcctctgctcctgccagtctgacttcaggtcagaactacagtgagttatctgggtggtctcctaagcagggacagatactaagcagaatccatgtctttgtcacctctgcagagaaccttgcctctaaaccgcgggctgtttggaggagtcccagcttctagacccccaccccaccctgaggagcagctccattaggtcacagagccccagactccttagtgagcacaaactgtcttcccagaggcctcaagcaggagtggggtcggggagcaggggtggacctcacagaacaggacagctagtccacaggggactccatgacagctgatcctcaggctgccactttggtggtacagatgagaagacatggttctatctctaagggctgctgtgaagtccttccccgcctcagctctggatatcctttggtgctataagcctatgttatgctatgcagtcttttggtgtcagtccaggacagtagtctatggacagtcaaaacagcaagtggagtcttagctcactggaagtcatctcagctacccctcttacctcgggacttcgctggaagagatgtggtttggtaccctttgttagggtggcagagatagctgcttttcctgcagtactgaatagcaagctttgcctctttgctataagcaacccctaaacgtcaggaaccttcctttttctaaatctgagatccagagtggcatctttcatttctgttgttctaaggtttcccaagtcacttcctttaggaagcaggtggcacttgcctggtccatatgcaaatctcaactctcctggtgcctgctattcagttctcccagccaaagctttggacagggtctaattacaacctgtggctagcaacctccttgtggggtctaacaactctttcacagaggtcacccaagaccatcagaaaactcagatatttacattttgattcataacagtagcaaaattacagttatgaaatagtaatgaaaataatttgtggttgagggtcaccacaatatgaggaacggtattcaagggtcacagcatgaggaaagttgaaaaccactgctccagttaaacctcttctgctgctgccagggacagccaagcagaccaggctcagtggtgccgctgtagtgccttctttttccagcccggcccaggctcattacctcagacagatggcttccccaagataagctgttggtgacagagtctctctaacaagctgaatgggaagttcctcccagtgtataggggtttctctctaccacctcagcttcctgtgaaacatggccacagggactggctgagggctggctctgcctcttcaggatgtcagaggttggctgaagggtcagactctctctgtgcccttgactttacagagacagaggtcgctccagaaacggaggcatgtgatgagctctagaaagtagcttacacccaaatctgtccgtaaggtttatagagcaaccaggcagacagaggctcacagactgtgtagatgcttagaggttctaaatttggtttaaagactgttatgtgaaatgcattctaatccccctgtcttggcagatatacctcctaaaagtggggttcaaacttagggttttaggctcctttgggggctagttttatctgaccaagtgtttctgcttgactgctccatccctgactccacccacgggtgcttctcttgagccatgtgcatatgcacggcagcttacttttgggcatggtaccggggctctgatgaattttggaagcagaatcaaggcatttggacaaggaatctttgctctagtagagaatggatgacgggcgtagctccagccctaacctgcagggctcagagcagggaccactcttcgctagctctgagagggggccacggctagtgtgtgcatcctaactgcaaactcagggctctttctcatcaccagtcatctcatctcatgtctttttatttttttattttattttattttattttattttattttgattttgatttttcgagacagggtttctctgtatagccctggctgtcctggaactcactttgtagactaggctggcctcgaactcaaaaatccgcctgcctctgcctcccaagtgctgggattaaaggtgtgcgccaccacacccagctcatctcatgtcttaaatgccagaaagccaacatgggccaagccagtgagggctccaagtgtgggctctggaaggaccatggtcatctgagattgttgaggactcaggagctgctgttactgggaccgaatgagtgcatccccctgacagccagagaacctgcctgcttggtctcaaaaatactaaccctgcacagatctcgtgaccacccctcccccagccccaggccagcacacaagttcacactatggtcagagtgagctttgtatcccttgttctcttcaccaccaggccagagtggcagtggggataaggagagcactctagagtctgagggcaggagacttgagataagccttggttctatatgctctcgggcttgtcctaaactgttcccagtgtcctcatctgcgagt

SEQ ID NO: 7 - DNA, 27 bp, Artificial
  Oligonucleotide Primer
tggtgcttagcatctgtcaaggtgtag

SEQ ID NO: 8 - DNA, 27 bp, Artificial
  Oligonucleotide Primer
ctacactgtatttcagagcctggtgag

SEQ ID NO: 9 - DNA, 27 bp, Artificial
  Oligonucleotide Primer
aggctctgaaatacagtgtaggtggtc

SEQ ID NO: 10 - DNA, 38 bp, Artificial
  Oligonucleotide Primer
aaagcggccgcggaggtagcatcccatagaaaggcaag

SEQ ID NO: 11 - DNA, 35 bp, Artificial
  Oligonucleotide Primer
tttactagttggacagcagcgacacgccgatagtg

SEQ ID NO: 12 - DNA, 38 bp, Artificial
  Oligonucleotide Primer
aaaggcgcgcctgttggtggacatacaccccaggtgag

SEQ ID NO: 13 - DNA, 38 bp, Artificial
  Oligonucleotide Primer
tttggccggccggtcccagtaacagcagctcctgagtc

SEQ ID NO: 14 - DNA, 27 bp, Artificial
  Oligonucleotide Primer
agccagagaacctgcctgcttggtctc

SEQ ID NO: 15 - DNA, 27 bp, Artificial
  Oligonucleotide Primer
actcgcagatgaggacactgggaacag

SEQ ID NO: 16 - DNA, 27 bp, Artificial
  Oligonucleotide Primer
gctcggcttccaccagctatatgcctc

SEQ ID NO: 17 - DNA, 27 bp, Artificial
  Oligonucleotide Primer
actcacctggggtgtatgtccaccaac

SEQ ID NO: 18 - DNA, 27 bp, Artificial
  Oligonucleotide Primer
gtcgtgacccatggcgatgcctgcttg

SEQ ID NO: 19 - DNA, 27 bp, Artificial
  Oligonucleotide Primer
catgccgcctgcgccctattgatcatg

(19) United States
(12) Patent Application Publication
(10) Pub. No.: US 2009/0048200 A1
(43) Pub. Date: Feb. 19, 2009

(54) GPR 26 G-Protein Coupled Receptor

(76) Inventors: Alan Hendrick, Cambridge (GB); John Dixon, Cambridge (GB); Tere Michelle Tait, Cambridge (GB); Rosa Fradley, Cambridge (GB); Malcolm Sheardown, Cambridge (GB); Haiping Liu, Chromos (SG); Kok Siu Foo, Chromos (SG)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 12/090,741

(22) PCT Filed: Oct. 30, 2006
(86) PCT No.: PCT/GB2006/004024

§ 371 Date: Aug. 4, 2008

(30) Foreign Application Priority Data
Oct. 28, 2005 (GB) 0522077.7
Mar. 8, 2006 (GB) 0604684.1

(51) Int. Cl. A61K 31/7088; G01N 33/00; C12Q 1/02; A61P 25/00; A01K 67/027; C12Q 1/68

(52) U.S. Cl. 514/44; 436/86; 435/29; 800/3; 800/13; 435/6; 800/9

(57) ABSTRACT

We disclose GPR26 G-protein coupled receptor (GPCR) polypeptides comprising the amino acid sequence shown in SEQ ID NO. 3 or SEQ ID NO: 5, and homologues, variants and derivatives thereof. Nucleic acids capable of encoding GPR26 polypeptide are also disclosed. The GPR26 receptor is useful in the diagnosis and therapy of mental illness.

### FIELD OF THE INVENTION

This invention relates to G-protein coupled receptor (GPCR) polypeptides and nucleic acids, hereinafter referred to as “GPR26 GPCR”, which is a member of the family of GPCRs. The invention also relates to inhibiting or activating the action of such nucleic acids and polypeptides, in particular to provide candidates for the therapy of mental illness.

### BACKGROUND TO THE INVENTION

Many medically significant biological processes are mediated by proteins participating in signal transduction pathways that involve G-proteins and/or second messengers, for example, cAMP (Lefkowitz, Nature, 1991, 351: 353-354). These proteins are referred to as “proteins participating in pathways with G-proteins” or “PPG proteins”. Examples of PPG proteins include the GPCRs, such as those for adrenergic agents and dopamine (Kobilka, B. K., et al., Proc. Natl Acad. Sci., USA, 1987, 84: 46-50; Kobilka B. K., et al., Science, 1987, 238: 650-656; Bunzow, J. R., et al., Nature, 1988, 336: 783-787), G-proteins themselves, effector proteins, for example, phospholipase C, adenyl cyclase, and phosphodiesterase, and actuator proteins, for example, protein kinase A and protein kinase C (Simon, M. I., et al., Science, 1991, 252: 802-8).

In one form of signal transduction, hormone-binding activates adenylate cyclase inside the cell. A G-protein connects the hormone receptor to adenylate cyclase. G-protein exchanges GTP for bound GDP when activated by a hormone receptor. The GTP-bound form then binds to and activates adenylate cyclase. Hydrolysis of GTP to GDP, catalysed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of GPCRs is characterised by seven putative transmembrane domains, which are believed to represent transmembrane α-helices connected by extracellular or cytoplasmic loops. GPCRs (also known as 7TM receptors) include a wide range of biologically active receptors, such as hormone, viral, growth factor and neuroreceptors. The seven conserved hydrophobic stretches of about 20 to 30 amino acids, connect at least eight divergent hydrophilic loops. The 7 transmembrane regions are designated as TM1 to TM7, respectively. TM3 has been implicated in signal transduction. The GPCR family includes dopamine receptors which bind to neuroleptic drugs used for treating psychotic and neurological disorders. Other members of this family include, but are not limited to, calcitonin, adrenergic, endothelin, cAMP, adenosine, muscarinic, acetylcholine, serotonin, histamine, thrombin, kinin, follicle stimulating hormone, opsins, endothelial differentiation gene-1, rhodopsins, odorant, and cytomegalovirus receptors.

Most GPCRs have single conserved cysteine residues in each of the first two extracellular loops, which form disulphide bonds that are believed to stabilise protein structure. Phosphorylation and lipidation (pamitylation or farnesylation) of cysteine residues can influence signal transduction of some GPCRs. Most GPCRs contain potential phosphorylation sites within the third cytoplasmic loop and/or the carboxy terminus. For several GPCRs, such as the β-adrenoreceptor, phosphorylation by protein kinase A and/or specific receptor kinases mediates receptor desensitization. For some receptors, the ligand binding sites of GPCRs are believed to comprise hydrophilic sockets formed by several GPCR transmembrane domains, the sockets being surrounded by hydrophobic residues of the GPCRs. The hydrophilic side of each G-protein coupled receptor transmembrane helix is thought to face inward and form a polar ligand binding site. TM3 has been implicated in several G-protein coupled receptors as having a ligand binding site, such as the TM3 aspartate residue. TM5 serines, a TM6 asparagine and TM6 or TM7 phenylalanines or tyrosines are also implicated in ligand binding.

GPCRs can be intracellularly coupled by heterotrimeric G-proteins to various intracellular enzymes, ion channels and transporters (see, Johnson et al., Endoc. Rev., 1989, 10: 317-331). Different G-protein α-subunits preferentially stimulate particular effectors to modulate various biological functions in a cell. Phosphorylation of cytoplasmic GPCR residues is an important mechanism for the regulation of G-protein coupling of some GPCRs. GPCRs are found in numerous sites within a mammalian host. Nearly 350 therapeutic agents targeting GPCRs have been successfully introduced onto the market.

GPCRs have an established, proven history as therapeutic targets. Clearly there is a need for identification and characterization of further receptors which can play a role in preventing, ameliorating or correcting disorders, dysfunctions and diseases.

Mental illness is a broad term used to define brain disorders, in particular those involving affective or emotional instability, behavioural dysreguation and/or cognitive impairment. Mental illness can be debilitating to the sufferer and caring for one suffering is a significant emotional and financial burden.

Anxiety is defined as ‘the protective physiological and behavioural response of a subject to potential threats that may impair its homeostasis’. It is considered pathological when this response is excessive or maladaptive, and becomes disabling (Beizung, C. & Griebel, G (2001). ‘Measuring normal and pathological anxiety-like behaviour in mice: a review’. Behav Brain Res 125, 141-9). There are six types of anxiety disorders: Generalised anxiety disorder, marked by excessive worry in multiple areas; post-traumatic stress disorder, characterised by intrusive, anxiety-provoking memory of trauma; specific phobias, notable for intense fear of a specific trigger; Social anxiety disorder, involving fear and avoidance of social situations; Panic disorder, characterised by unpredictable, rapid attacks of intense anxiety; Obsessive compulsive disorder, marked by anxious obsessions and anxiety-reducing compulsive behaviours (Gordon, J. A. & Hen, R. (2004). ‘Genetic approaches to the study of Anxiety’. Annu Rev Neurosci. 27, 193-222). Neural circuitry involving the amygdala and hippocampus is thought to underlie anxiety (Rosen, J. B. & Schulkin, J. (1998): “From normal fear to pathological anxiety”. Psychological Review. 105(2); 325-350). Anxiety disorders are the most common mental illnesses.

Depression can be described as ‘the manifestation of incapacity to cope with various lifetime stressors, on the psychological, behavioural and physiological levels’ (Cryan, J. F & Mombereau, C. (2004). ‘In search of a depressed mouse: utility of models for studying depression-related behaviour in genetically modified mice’. Mol. Psychiatry. 9, 326-57). It is characterised by feelings of sadness accompanied by emotional and physical withdrawal (Taylor, C. Fricker, A. D., Devi, L. A and Gomes, I. (2005). ‘Mechanisms of action of antidepressants: from neurotransmitter systems to signalling pathways’. Cell Signal. 17, 549-57). Depression is not a single disease, but comprises numerous pathologies. Although subtypes have been defined, they are based on symptomatic differences and may not reflect underlying disease states (Nestler, E. J. et al. (2002). ‘Neurobiology of depression’. Neuron. 34, 13-25). Current antidepressants are inefficient in ˜40% of sufferers.

Psychosis is a term defining a mental state having severely impaired thought and perception. A psychotic episode is debilitating and commonly involves hallucinations, paranoia, delusions, disorganised thinking and impairment of social interaction. Psychoses generally involve a loss of contact with reality, which can be highly destructive to the sufferer. Psychoses manifest in conditions including bipolar disorder, severe clinical depression and schizophrenia. These conditions are not well understood and effective therapies to prevent and treat them are required. In particular, there is a significant unmet need in the therapy of the negative symptoms of schizophrenia. The most commonly used drugs, clozapine and olanzapine, take 2-4 weeks to be fully efficacious and have relatively weak effects on the negative symptoms of schizophrenia.

There is a significant overlap in the symptoms of anxiety, depression and psychosis, in particular depression and anxiety can be co-occurring (occurring together independently and without mood congruence) or comorbid (occurring together with overlapping symptoms and with mood congruence). Furthermore, it is thought that 50% of psychotics have concurrent anxiety.

There remains a strong need for effective therapies to prevent and treat mental illness, in particular anxiety disorders, disorders of psychosis and depressive disorders.

### SUMMARY OF THE INVENTION

The present invention is based on the surprising realisation that GPR26 is implicated in mental illness and is therefore a useful therapeutic and diagnostic target. The inventors have found that, surprisingly, animals lacking a functional GPR26 gene demonstrate phenotypes indicating mental illness. Therefore, the GPR26 receptor is implicated in mental illness and is a therapeutic and diagnostic target.

According to a first aspect of the invention, a method for identifying an agent suitable for therapy of mental illness comprises the step of determining whether a candidate agent affects the activity of GPR26.

According to a second aspect of the invention, a non-human transgenic animal has a functionally disrupted endogenous GPR26 gene.

According to a third aspect of the invention, a non-human transgenic animal having a functionally disrupted endogenous GPR26 gene is used to identify an agonist or antagonist of GPR26 for the therapy of mental illness.

According to a fourth aspect of the invention, the non-human transgenic animal having a functionally disrupted endogenous GPR26 gene is used as a model for mental illness.

According to a fifth aspect of the invention, a method for determining the presence or susceptibility of mental illness in an individual comprises the steps of (i) determining the expression level of GPR26 gene in a sample isolated from the patient and (ii) determining, compared to a control, whether the individual has or is susceptible to mental illness.

According to a sixth aspect of the invention, an agent that affects the activity of GPR26 polypeptide is used in the manufacture of a medicament for the prevention or treatment of mental illness.

According to a seventh aspect of the invention, an exogenous GPR26 polynucleotide is used in the manufacture of a medicament for the treatment or prevention of mental illness.

According to an eighth aspect of the invention, an agent that increases or decreases the expression of a GPR26 polynucleotide is used in the manufacture of a medicament for the treatment or prevention of mental illness.

### BRIEF DESCRIPTION OF THE DRAWINGS

The invention is described with reference to the following figures, wherein:

FIG. 1 is an alignment of human GPR26 with Human GPR78 (Identities=170/327 (51%), Positives=222/327 (67%), Gaps=4/327 (1%));

FIG. 2 shows the results of a POPMAP test, indicating that GPR26 knockout mice display a reduced locomotor activity compared to wild type control mice;

FIG. 3 shows the results of a LABORAS test showing reduced locomotor activity (locomotion duration—FIG. 3a, distance traveled—FIG. 3b) in GPR 26 knockout mice compared to wild type control mice;

FIG. 4 shows the results of a Porsolt Forced Swim test;

FIG. 5 shows the response of GPR26 knockout and wild type mice to phencyclidine;

FIG. 6 shows the genomic sequence of mouse GPR26 (Sequence Range: 1 to 6731, also included as SEQ ID NO: 6) indicating the location of the primers used in the targeting strategy;

FIG. 7 is a diagram showing the knockout vector;

FIG. 8 is a graph showing the results of the open field for knockout (KO) compared to wildtype (WT) mice (Z3=central zone);

FIG. 9 is a graph showing the results of the elevated zero maze for knockout (KO) compared to wildtype (WT) mice (P.Time=time spent in arm);

FIG. 10 is a graph showing the results of the elevated plus maze for knockout (KO) compared to wildtype (WT) mice;

FIG. 11 is a graph showing the results of the MK-801-induced hyperlocomotion for knockout (KO) compared to wildtype (WT) mice;

FIG. 12 depicts graphs showing swim speed and resting time as observed in the morris water maze; and

FIG. 13 shows the PPI inhibition data, where * denotes significant (p<0.05) effect of drug treatment compared to equivalent vehicle treated group (paired T-test), and + denotes significant (p<0.05) effect of genotype compared to equivalent wild-type group (repeated measure ANOVA).

### SEQUENCE LISTINGS

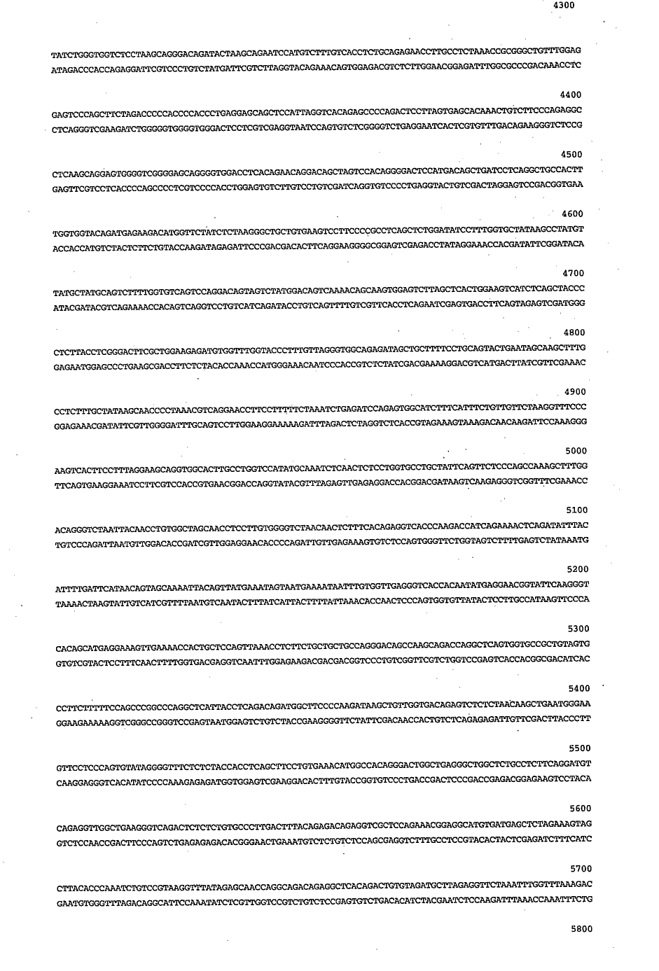

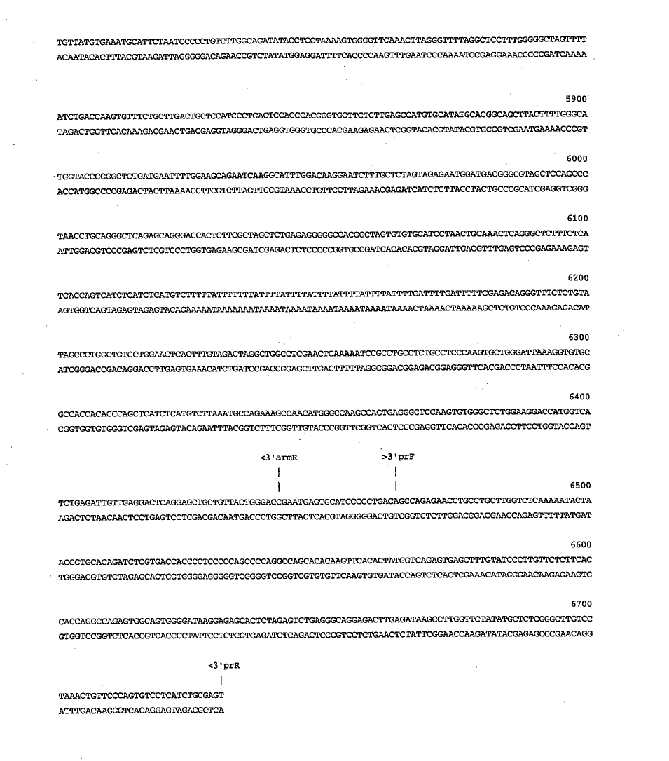

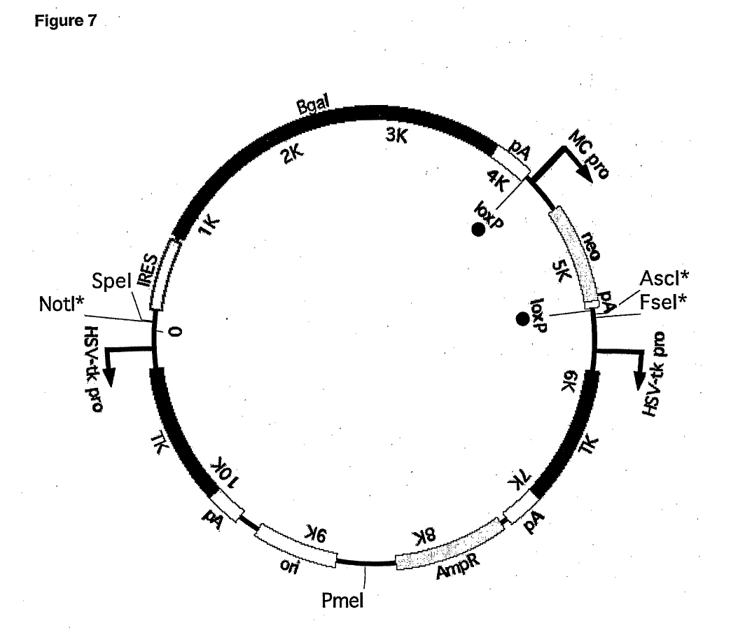

SEQ ID NO: 1 shows the cDNA sequence of human GPR26. SEQ ID NO: 2 shows an open reading frame derived from SEQ ID NO: 1. SEQ ID NO: 3 shows the amino acid sequence of human GPR26. SEQ ID NO: 4 shows the open reading frame of a cDNA for mouse GPR26. SEQ ID NO: 5 shows the amino acid sequence of mouse GPR26. SEQ ID NO: 6 (also shown as FIG. 6) shows the genomic sequence of mouse GPR26 from the 5′ arm to the 3′ arm. SEQ ID NO: 7 to 19 shows the nucleotide sequence of mouse GPR26 primers used in construction and analysis of the targeting vector and agents containing the targeted locus.

### DETAILED DESCRIPTION OF THE INVENTION

### GPR26 GPCR

The invention relates to a novel GPCR, in particular a rhodopsin type of 7 transmembrane receptor which we refer to as GPR26 GPCR, as well as homologues, variants or derivatives thereof.

GPR26 is structurally related to other proteins of the G-protein coupled receptor family, as shown by the results of sequencing the amplified cDNA products encoding human GPR26. The cDNA sequence of SEQ ID NO: 1 contains an open reading frame (SEQ ID NO: 2, nucleotide numbers 54 (including start codon) to 1067 (including stop codon)) encoding a polypeptide of 337 amino acids shown in SEQ ID NO: 3. Human GPR26 is found to map to chromosomal location 10q26.2-q26.3.

### Identities and Similarities to GPR26

Analysis of the GPR26 polypeptide (SEQ ID NO: 3) using the HMM structural prediction software of pfam (http://www.sanger.ac.uk/Software/Pfam/search.shtml) confirms that GPR26 peptide is a GPCR of the 7TM-1 structural class.

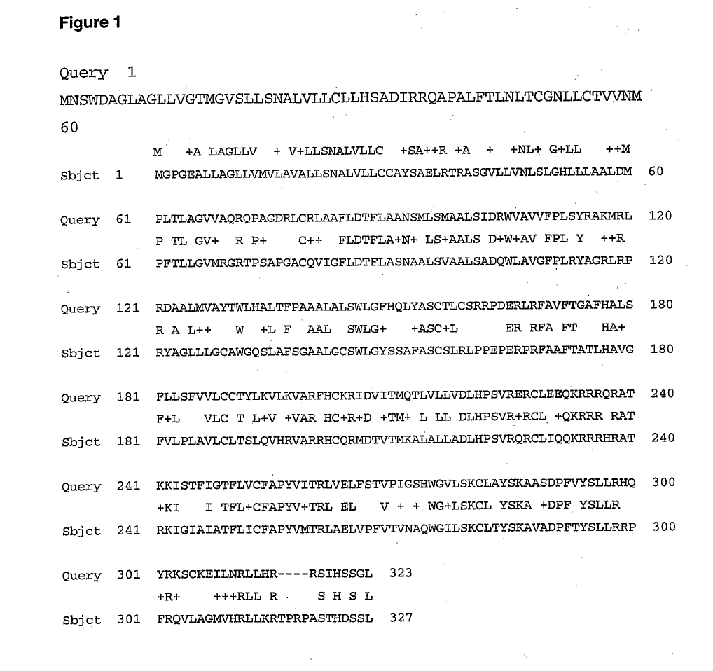

The closest human homologue, GPR78 (which lacks a mouse orthologue), shares 51% identity and 67% similarity with human GPR26 (see FIG. 1). Apart from GPR78, GPR26 does not belong to a subfamily of GPCRs, sharing the highest identity with gastrin releasing hormone BB2 receptor and serotonin 5-HT-5A and -5B receptors (25% and 23%, respectively).

The mouse homologue of the human GPR26 GPCR has been cloned, and its nucleic acid sequence and amino acid sequence are shown as SEQ ID NO: 4 and SEQ ID NO: 5 respectively. The mouse GPR26 GPCR cDNA of SEQ ID NO: 4 shows a high degree of identity with the human GPR26 GPCR (SEQ ID NO: 2) sequence, while the amino acid sequence (SEQ ID NO: 5) of mouse GPR26 GPCR shows a high degree of identity and similarity with human GPR26 GPCR (SEQ ID NO: 3). The GPR26 amino acid sequence shares 95% identities and 97% positives with its mouse orthologue. The mouse gene is located on chromosome 7 F3.

Human GPR26 is also 95% identical to its rat orthologue.

Human and mouse GPR26 GPCR are therefore members of a large family of GPCRs.

### Expression Profile of GPR26

Polymerase chain reaction (PCR) amplification of GPR26 cDNA detects expression of GPR26 in the brain only. In the mouse brain, GPR26 is expressed broadly in the CNS including the hippocampus, striatum, thalamus, forebrain, cortex, pons and the hypothalamus. Using GPR26 cDNA of SEQ ID NO: 1 to search the human and mouse EST data sources by BLASTN, high identity matches are found in cDNA derived from libraries originating from human infant brain and mouse 7 day neonate cerebellum, mouse 16 day neonate cerebellum, mouse visual cortex, mouse 10 day neonate cortex, mouse neonate brain and mouse embryo brains at 12.5, 15.5 and 17.5 days post conception. This indicates that GPR26 is expressed in these tissues. Accordingly, the GPR26 polypeptides, nucleic acids, probes, antibodies, expression vectors and ligands are useful for detection, diagnosis, treatment and other assays for diseases associated with over-, under- and abnormal expression of GPR26 GPCR in these and other tissues.

### GPR26 Associated Diseases

According to the methods and compositions described here, GPR26 is useful in therapy and diagnosis, for treating and diagnosing a range of diseases. GPR26 is also useful in assaying molecules that modify the activity of GPR26, as discussed below. A molecule that modifies GPR26 is a potential therapeutic agent for a GPR26 associated disease. Diseases in which GPR26 is implicated are referred to for convenience as GPR26 associated diseases. These diseases are mental illness. As used herein, the term mental illness refers to disorders of brain function, including illnesses that include affective or emotional instability, behavioural dysregulation and/or cognitive dysfunction or impairment, such as anxiety disorders, psychoses and depressive disorders. The term “mental illness” also includes disorders associated with a locomotor deficit, i.e. movement disorders, such as Parkinson's Disease, Huntington's Chorea and disorders involving ataxia. Human and animal (veterinary) therapy and diagnosis is within the scope of the invention.

Human GPR26 maps to chromosomal location 10q26.2-q26.3. Accordingly, in a specific embodiment, GPR26 GPCR may be used to treat or diagnose a disease which maps to this locus, chromosomal band, region, arm or the same chromosome.

The data provided herein indicate that GPR26 is implicated in mental illness, as detailed in the examples and summarised below. In particular, the data indicate that GPR26 knock-out mice demonstrate anxiety, depression and psychosis.

In the human RT-PCR panel, the gene is expressed in the brain only; in the mouse brain RT-PCR panel, the gene is expressed broadly in the CNS. LacZ staining also indicates an expression pattern supporting GPR26 involvement in psychiatric disease, recognition and memory (example 3(I)). Behavioural tests (example 3(II)) show that GPR26 knock-out mice display reduced locomotor activity compared to wild-type mice, with no change in collateral behaviour. The reduced locomotor activity is of CNS (and not muscular) origin. GPR26 knock-out mice also cope better in a stressful environment than wild-type mice, and show less depression.

Open Field, elevated plus maze and elevated zero maze tests (examples 4-6) indicate that GPR26 knock-out mice are anxious.

An MK-801-induced hyperlocomotion (example 7) test indicates that GPR26 knock-out mice have a psychogenic phenotype.

A watermaze test (example 8) also indicates that GPR26 mice have a locomotor deficit. The data in examples 4-6 and 8 also indicate that an agonist acting at GPR26 would be useful in treating a range of movement disorders, preferably Parkinson's Disease or Huntington's Chorea.

Pre-pulse inhibition data (example 9) support the psychosis phenotype seen in example 7 and indicate that modulation of GPR26, preferably by an agonist, has utility in the treatment of psychoses, preferably schizophrenia.

GPR26 associated diseases are mental illnesses, as defined above. Preferred diseases include anxiety disorders, diseases involving psychosis and depressive disorders. The data indicate that, in a preferred embodiment, an agonist to GPR26 may be beneficial as an anxiolytic drug and/or an anti-psychotic drug. In an alternative preferred embodiment, an antagonist to GPR26 is useful as an anti-depressant drug, or in treating ADHD (Attention Deficit Hyperactivity Disorder).

As used herein, the term “anxiety disorder” refer to disorders involving pathological anxiety. Anxiety is ‘the protective physiological and behavioural response of a subject to potential threats that may impair its homeostasis’. It is considered pathological when this response is excessive or maladaptive, and becomes disabling (Belzung, C. & Griebel, G (2001, supra). There are six types of anxiety disorders: Generalised anxiety disorder, marked by excessive worry in multiple areas; post-traumatic stress disorder, characterised by intrusive, anxiety-provoking memory of trauma; specific phobias, notable for intense fear of a specific trigger; Social anxiety disorder, involving fear and avoidance of social situations; Panic disorder, characterised by unpredictable, rapid attacks of intense anxiety; Obsessive compulsive disorder, marked by anxious obsessions and anxiety-reducing compulsive behaviours (Gordon, J. A. & Hen, R. (2004), supra. Neural circuitry involving the amygdala and hippocampus is thought to underlie anxiety (Rosen, J. B. & Schulkin, J. (1998), supra).

As used herein, the term “psychosis” refers to a disease in which psychosis is, or can be, a symptom. Psychosis is generally considered to be a symptom of severe mental illness, but is not a diagnosis in itself. Any mental illness that is or can be associated with psychosis is within the scope of the invention. Preferred diseases involving psychosis are schizophrenia, bipolar disorder (manic depression) and severe clinical depression. Brain injury (or other neurological disorder), drug intoxication and withdrawal (especially alcohol, barbiturates and benzodiazepenes), lupus, electrolyte disorders in the elderly (such as urinary tract infections), pain syndromes, sleep depression and extreme stress (such as post-traumatic stress disorder) can also cause a psychotic episode, and are within the scope of the invention. The most preferred psychosis is schizophrenia.

As used herein, the term “depressive disorder” refers to diseases involving depression, which is ‘the manifestation of incapacity to cope with various lifetime stressors, on the psychological, behavioural and physiological levels’ (Cryan, J. F & Mombereau, C. (2004), supra) It is characterised by feelings of sadness accompanied by emotional and physical withdrawal (Taylor, C et al (2005), supra).

There is a significant overlap in the symptoms of anxiety, depression and psychosis, in particular depression and anxiety can be co-occurring (occurring together independently and without mood congruence) or comorbid (occurring together with overlapping symptoms and with mood congruence).

Further preferred diseases are disorders associated with a locomotor deficit, i.e. movement disorders, such as Parkinson's Disease, Huntington's Chorea and disorders involving ataxia. Movement disorders associated with the basal ganglia, such as Parkinson's Disease, are preferred.

Preferred mental illnesses include major depression, dysthymia, bipolar disorder, seasonal affective disorder, post natal depression, Social anxiety, post traumatic stress disorder, phobias, social phobia, special phobias, panic disorder, obsessive compulsive disorder, acute stress, disorder, separation anxiety disorder, generalised anxiety disorder, psychoses such as schizophrenia, bipolar disorder (manic depression) and severe clinical depression, psychoses associated with other conditions (including brain injury (or other neurological disorder), drug intoxication and withdrawal (especially alcohol, barbiturates and benzodiazepenes), lupus, electrolyte disorders in the elderly (such as urinary tract infections), pain syndromes, sleep depression and extreme stress (such as post-traumatic stress disorder)), drug abuse, Alzheimer's disease, ishaemic/vascular dementia, Pick's disease, diffuse Lewy body dementia, frontotemporal dementias, corticobasal degeneration, Huntington's disease, progressive supranuclear palsy, AIDS/HIV dementia, prion infections, encephalitis, ADHD (Attention Deficit Hyperactivity disorder), neurosyphilis, vasculitis and progressive multifocal leukoencephalopathy

### Methods Employed

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### GPR26 GPCR Polypeptides

As used here, the term “GPR26 GPCR polypeptide” refers to a polypeptide comprising the amino acid sequence shown in SEQ ID No. 5 or more preferably SEQ ID NO: 3, or a homologue, variant or derivative thereof. “Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. A given polypeptide may contain many types of modifications.

Polypeptides may be branched as a result of ubiquitination, and they may be cyclic, with or without branching. Cyclic, branched and branched cyclic polypeptides may result from post-translation natural processes or may be made by synthetic methods. Many protein modifications are known, see, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et aL, “Protein Synthesis: Posttranslational Modifications and Aging”, Ann NY Acad Sci (1992) 663:48-62.

The terms “variant”, “homologue”, “derivative” or “fragment” as used herein include any substitution, variation, modification, replacement, deletion or addition of one (or more) amino acid from or to a sequence. The variant may have a deletion, insertion or substitution variation that produces a silent change and a functionally equivalent polypeptide. Deliberate amino acid substitutions may be made on the basis of similar physio-chemical properties such as size, charge and hydrophobicity.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Unless the context admits otherwise, references to “GPR26” and “GPR26 GPCR” include references to such variants, homologues, derivatives and fragments of GPR26.

Preferably, as applied to GPR26, the amino acid sequence has GPCR activity, more preferably having at least the same activity of the GPR26 GPCR shown as SEQ ID NO: 3 or SEQ ID NO: 5. In particular, the term “homologue” covers identity with respect to structure and/or function providing the resultant amino acid sequence has GPCR activity. With respect to sequence identity (i.e. similarity), preferably there is at least 60%, 70%, 75%, 80%, 85%, 90%, 95% or, most preferably there is at least 95%, more preferably at least 98%, sequence identity. These terms also encompass polypeptides derived from amino acids which are allelic variations of the GPR26 GPCR nucleic acid sequence.

Where reference is made to the “receptor activity” or “biological activity” of a receptor such as GPR26 GPCR, these terms are intended to refer to the metabolic or physiological function of the GPR26 receptor, including similar activities or improved activities or these activities with decreased undesirable side effects. Also included are antigenic and immunogenic activities of the GPR26 receptor. Examples of GPCR activity, and methods of assaying and quantifying these activities, are known in the art, and are described in detail elsewhere in this document.

GPR26 polypeptides of the invention may further comprise heterologous amino acid sequences, typically at the N-terminus or C-terminus, preferably the N-terminus. Heterologous sequences may include sequences that affect intra or extracellular protein targeting (such as leader sequences). Heterologous sequences may also include sequences that increase the immunogenicity of the polypeptide of the invention and/or which facilitate identification, extraction and/or purification of the polypeptides. Another heterologous sequence that is particularly preferred is a polyamine acid sequence such as polyhistidine which is preferably N-terminal. A polyhistidine sequence of at least 10 amino acids, preferably at least 17 amino acids but fewer than 50 amino acids is especially preferred.

The GPR26 GPCR polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

GPR26 polypeptides of the invention are preferably made by recombinant means, using known techniques. However they may also be made by synthetic means using techniques well known to skilled persons such as solid phase synthesis. Polypeptides of the invention may also be produced as fusion proteins, for example to aid in extraction and purification. Examples of fusion protein partners include glutathione-S-transferase (GST), 6×His, GAL4 (DNA-binding and/or transcriptional activation domains) and β-galactosidase. It may also be convenient to include a proteolytic cleavage site between the fusion protein partner and the protein sequence of interest to allow removal of fusion protein sequences, such as a thrombin cleavage site. Preferably the fusion protein will not hinder the function of the protein of interest sequence.

GPR26 polypeptides of the invention may be in a substantially isolated form. This term is intended to refer to alteration by the hand of man from the natural state. If an “isolated” composition or substance occurs in nature, it has been changed or removed from its original environment, or both. For example, a polynucleotide, nucleic acid or a polypeptide naturally present in a living animal is not “isolated,” but the same polynucleotide, nucleic acid or polypeptide separated from the coexisting materials of its natural state is “isolated”, as the term is employed herein.

It will however be understood that the GPR26 GPCR protein may be mixed with carriers or diluents which will not interfere with the intended purpose of the protein and still be regarded as substantially isolated. A polypeptide of the invention may also be in a substantially purified form, in which case it will generally comprise the protein in a preparation in which more than 90%, for example, 95%, 98% or 99% of the protein in the preparation is a GPR26 GPCR polypeptide of the invention.

The present invention also relates to peptides comprising a portion of a GPR26 polypeptide according to the invention. Thus, fragments of GPR26 GPCR and its homologues, variants or derivatives are included. The peptides of the present invention may be between 2 and 200 amino acids, preferably between 4 and 40 amino acids in length. The peptide may be derived from a GPR26 GPCR polypeptide as disclosed here, for example by digestion with a suitable enzyme, such as trypsin. Alternatively the peptide, fragment, etc may be made by recombinant means, or synthesised synthetically.

The term “peptide” includes the various synthetic peptide variations known in the art, such as a retroinverso D peptides. The peptide may be an antigenic determinant and/or a T-cell epitope. The peptide may be immunogenic in vivo. Preferably the peptide is capable of inducing neutralising antibodies in vivo.

The GPR26 polypeptides according to the invention may comprise a sequence which is conserved across species. A conserved region shows a high degree of homology between at least two species. For example, the region may show at least 60%, preferably at most 70%, preferably at least 80%, more preferably at least 90%, even more preferably at least 95% identity at the amino acid level using the tests described above. Peptides which comprise a sequence which corresponds to a conserved region may be used in therapeutic strategies as explained in further detail below. Alternatively, the GPR26 GPCR peptide may comprise a sequence which corresponds to at least part of a non-conserved region. A heterologous region shows a low degree of homology between at least two species.

### GPR26 GPCR Polynucleotides and Nucleic Acids

This invention encompasses GPR26 polynucleotides, GPR26 nucleotides and GPR26 nucleic acids, methods of production, uses of these, etc, as described in further detail elsewhere in this document.

The terms “GPR26 polynucleotide”, “GPR26 nucleotide” and “GPR26 nucleic acid” may be used interchangeably, and are intended to refer to a polynucleotide/nucleic acid comprising a nucleic acid sequence as shown in SEQ ID NO: 1, SEQ ID NO: 2 or SEQ ID NO: 4, or a homologue, variant or derivative thereof. Preferably, the polynucleotide/nucleic acid comprises or is a homologue, variant or derivative of the nucleic acid sequence SEQ ID NO: 1 or SEQ ID NO: 2, most preferably, SEQ ID NO: 2.

These terms are also intended to include a nucleic acid sequence capable of encoding a polypeptide and/or a peptide of the present invention, i.e., a GPR26 polypeptide. Thus, GPR26 GPCR polynucleotides and nucleic acids comprise a nucleotide sequence capable of encoding a polypeptide comprising the amino acid sequence shown in SEQ ID NO: 3 or SEQ ID NO: 5, or a homologue, variant or derivative thereof. Preferably, the GPR26 GPCR polynucleotides and nucleic acids comprise a nucleotide sequence capable of encoding a polypeptide comprising the amino acid sequence shown in SEQ ID NO: 3, or a homologue, variant or derivative thereof.

The term “Polynucleotide” is well known in the art and to refer to any polyribonucleotide or polydeoxyribonucleotide, which may be modified or unmodified RNA or DNA. “Polynucleotides” include, without limitation single- and double-stranded molecules (and molecules comprising double and single stranded regions), hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be RNA or (preferably) DNA, of genomic, synthetic or recombinant origin which may be double-stranded or single-stranded whether representing the sense or antisense strand or combinations thereof.

Preferably, the resultant nucleotide sequence encodes a polypeptide having GPCR activity, preferably having at least the same activity of the GPCR shown as SEQ ID NO: 3 or SEQ ID NO: 5. Preferably, the term “homologue” refers to a polynucleotide that encodes a polypeptide which has GPCR activity. With respect to sequence identity (i.e. similarity), preferably there is at least 60%, more preferably at least 70%, more preferably at least 75%, more preferably at least 85%, more preferably at least 90% sequence identity. More preferably there is at least 95%, more preferably at least 98%, sequence identity. These terms also encompass allelic variations of the sequences.

### Calculation of Sequence Homology

Sequence identity with respect to any of the sequences presented here can be determined by a simple “eyeball” comparison (i.e. a strict comparison) of any one or more of the sequences with another sequence to see if that other sequence has, for example, at least 70% sequence identity to the sequence(s).

Relative sequence identity can also be determined by computer programs that can calculate % identity between two or more sequences using any suitable algorithm for determining identity, using for example default parameters. Examples of such a computer program include CLUSTAL, the GCG program package (Devereux et al 1984 Nucleic Acids Research 12: 387), FASTA (Atschul et al 1990 J Molec Biol 403-410), and BLAST (Ausubel et al, 1999 ibid).

Preferably, the BLAST algorithm is employed, provided at http://www.ncbi.nlm.nih.gov/BLAST, with parameters set to default values (Histogram=yes; descriptions=100; Expect—10; Alignments=50; matrix—BLOSUM62 (no alternative scoring matrices for BLASIN); Filter—Dust (BLASIN), SEG (other programs)). The BLAST algorithm is described in detail at http://www.ncbi.nih.gov/BLAST/blast_help.html, which is incorporated herein by reference.

### Hybridisation and Homologues

The present invention encompasses nucleotide sequences that are capable of hybridising to the sequences presented herein, or any fragment or derivative thereof, or to the complement of any of the above. Preferably, the sequence capable of hybridising is the complement of the relevant sequence.

Nucleotide sequences of the invention capable of selectively hybridising to the nucleotide sequences presented herein, or to their complement, will be generally at least 60%, preferably at least 70%, more preferably at least 75%, more preferably at least 85 or 90% and even more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to SEQ ID NO: 1, 2 or 4, preferably at least 60%, preferably at least 70%, 80% or 90% and more preferably at least 95% homologous to one of the sequences.

The term “selectively hybridizable” means that the nucleotide sequence used as a probe is used under conditions where a target nucleotide sequence of the invention is found to hybridize to the probe at a level significantly above background. Also included within the scope of the present invention are nucleotide sequences that are capable of hybridizing to the nucleotide sequences presented herein under conditions of intermediate to maximal stringency in a process familiar to those skilled in the art. Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related nucleotide sequences.

In a preferred embodiment, the present invention covers nucleotide sequences that can hybridise to one or more of the GPR26 GPCR nucleotide sequences of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

The present invention also encompasses nucleotide sequences that are capable of hybridising to the sequences that are complementary to the sequences presented herein, or any fragment or derivative thereof. Likewise, the present invention encompasses nucleotide sequences that are complementary to sequences that are capable of hybridising to the sequence of the present invention. These types of nucleotide sequences are examples of variant nucleotide sequences. In this respect, the term “variant” encompasses sequences that are complementary to sequences that are capable of hydridising to the nucleotide sequences presented herein. Preferably, however, the term “variant” encompasses sequences that are complementary to sequences that are capable of hydridising under stringent conditions (eg. 65° C. and 0.1×SSC {1×SSC=0.15 M NaCl, 0.015 Na₃ citrate pH 7.0}).

### Cloning of GPR26 GPCR

The present invention enables the cloning of GPR26 GPCR, its homologues and other structurally or functionally related genes from human and other species such as mouse, pig, sheep, etc to be accomplished. Polynucleotides which hybridise to a nucleotide sequence contained in SEQ ID NO: 1, SEQ ID NO: 2, SEQ ID NO: 4 or a fragment thereof, may be used as hybridization probes for cDNA and genomic DNA, to isolate partial or full-length cDNAs and genomic clones encoding GPR26 GPCR from appropriate libraries. Such probes may also be used to isolate cDNA and genomic clones of other genes (including genes encoding homologues and orthologues from species other than human) that have sequence similarity, preferably high sequence similarity, to the GPR26 GPCR gene. Hybridization screening, cloning and sequencing techniques are known to those of skill in the art and are described in, for example, Sambrook et al (supra).

Typically nucleotide sequences suitable for use as probes are 60% identical, preferably 70% identical, preferably 80% identical, more preferably 90% identical, even more preferably 95% identical to that of the referent. The probes generally will comprise at least 15, preferably at least 30, and more preferably at least 50, nucleotides. Particularly preferred probes will range between 150 and 500 nucleotides, more particularly about 300 nucleotides.

One embodiment, to obtain a polynucleotide encoding a GPR26 GPCR polypeptide, including homologues and orthologues from species other than human, comprises the steps of screening an appropriate library under stringent hybridization conditions with a labelled probe having the SEQ ID NO: 1, SEQ ID NO: 2, SEQ ID NO: 4 or a fragment thereof and isolating partial or full-length cDNA and genomic clones containing said polynucleotide sequence. Such hybridization techniques are well known to those of skill in the art. Stringent hybridization conditions are as defined above or alternatively conditions under overnight incubation at 42 degrees C. in a solution comprising: 50% formamide, 5×SSC (150 mM NaCl, 15 mM trisodium citrate), 50 mM sodium phosphate (pH7.6), 5×Denhardt's solution, 10% dextran sulphate, and 20 microgram/ml denatured, sheared salmon sperm DNA, followed by washing the filters in 0.1×SSC at about 65 degrees C.

### Functional Assay for GPR26 GPCR

The cloned putative GPR26 GPCR polynucleotides may be verified by sequence analysis or functional assays. For example, the putative GPR26 GPCR or homologue may be assayed for receptor activity using methods of assaying receptor which activity are well known in the art. A preferred method is the Xenopus oocyte system using two electrode voltage clamps, as mentioned in Wagner et al, Cell Physiol Biochem. 2000; 10(1-2); 1-12, which is incorporated herein by reference. The Xenopus system may also be used to screen known ligands and tissue/cell extracts for activating ligands, as described in further detail below.

### Expression Assays for GPR26 GPCR

To design useful therapeutics for treating GPR26 GPCR associated diseases, it is useful to determine the expression profile of GPR26 (whether wild-type or a particular mutant). Methods known in the art may be used to determine the organs, tissues and cell types (as well as the developmental stages) in which GPR26 is expressed. For example, traditional or “electronic” (i.e. homology searching in a database) Northern blots (Sambrook, supra, ch. 7 and Ausubel, F. M. et al. supra, ch. 4 and 16.) may be conducted. Reverse-transcriptase PCR (RT-PCR) may also be employed to assay expression of the GPR26 gene or mutant. More sensitive methods for determining the expression profile of GPR26 include RNAse protection assays, as known in the art.

### Expression of GPR26 GPCR Polypeptides

The invention includes a process for producing a GPR26 GPCR polypeptide. The method comprises in general culturing a host cell comprising a nucleic acid encoding GPR26 GPCR polypeptide, or a homologue, variant, or derivative thereof, under suitable conditions (i.e., conditions in which the GPR26 GPCR polypeptide is expressed).

Methods which are well known to those skilled in the art are used to construct expression vectors containing sequences encoding GPR26 GPCR and appropriate transcriptional and translational control elements. These methods include in vitro recombinant DNA techniques, synthetic techniques, and in vivo genetic recombination. A variety of expression vector/host systems may be utilized to contain and express sequences encoding GPR26 GPCR. These will be apparent to the skilled person and include, but are not limited to, microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems in particular mammalian e.g. human cells, where a number of viral-based expression systems may be utilised. Artificial chromosomes (e.g. yeast, human) may be used to deliver large fragments of nucleic acid to a cell. The invention is not limited by the host cell employed.

The “control elements” or “regulatory sequences” are those non-translated regions of the vector (i.e., enhancers, promoters, and 5′ and 3′ untranslated regions) which interact with host proteins to carry out transcription and translation. Such elements may vary in their strength and specificity. Depending on the vector system and host utilized, any number of suitable transcription and translation elements, including constitutive and inducible promoters, may be used as will be apparent to one skilled in the art.

Specific initiation signals may also be used to achieve more efficient translation of sequences encoding GPR26 GPCR. Such signals include the ATG initiation codon and adjacent sequences. In cases where sequences encoding GPR26 GPCR and its initiation codon and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers appropriate for the particular cell system used, such as those described in the literature. (Scharf, D. et al. (1994) Results Probl. Cell Differ. 20:125-162.)

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, and WI38), are available from the American Type Culture Collection (ATCC, Bethesda, Md.) and may be chosen to ensure the correct modification and processing of the foreign protein.

For long term, high yield production of recombinant proteins, stable expression is preferred. For example, cell lines capable of stably expressing GPR26 GPCR can be transformed using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. Resistant clones of stably transformed cells may be proliferated using tissue culture techniques appropriate to the cell type. Any number of selection systems may be used to recover transformed cell lines, as is well known in the art.

Alternatively, host cells which contain the nucleic acid sequence encoding GPR26 GPCR and express GPR26 GPCR may be identified by a variety of other procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein sequences.

Host cells transformed with nucleotide sequences encoding GPR26 GPCR may be cultured under conditions suitable for the expression and recovery of the protein from cell culture. The protein produced by a transformed cell may be located in the cell membrane, secreted or contained intracellularly depending on the sequence and/or the vector used. As will be understood by those of skill in the art, expression vectors containing polynucleotides which encode GPR26 GPCR may be designed to contain signal sequences which direct secretion of GPR26 GPCR through a prokaryotic or eukaryotic cell membrane. Other constructions may be used to join sequences encoding GPR26 GPCR to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences, such as those specific for Factor XA or enterokinase (Invitrogen, San Diego, Calif.), between the purification domain and the GPR26 GPCR-encoding sequence may be used to facilitate purification. One such expression vector provides for expression of a fusion protein containing GPR26 GPCR and a nucleic acid encoding 6 histidine residues preceding a thioredoxin or an enterokinase cleavage site. The histidine residues facilitate purification on immobilized metal ion affinity chromatography (IMIAC; described in Porath, J. et al. (1992) Prot. Exp. Purif. 3: 263-281), while the enterokinase cleavage site provides a means for purifying GPR26 GPCR from the fusion protein. A discussion of vectors which contain fusion proteins is provided in Kroll, D. J. et al. (1993; DNA Cell Biol. 12:441-453).

Fragments of GPR26 GPCR may be produced not only by recombinant production, but also by direct peptide synthesis using solid-phase techniques. (Merrifield J. (1963) J. Am. Chem. Soc. 85:2149-2154.) Protein synthesis may be performed by manual techniques or by automation. Automated synthesis may be achieved, for example, using the Applied Biosystems 431A peptide synthesizer (Perkin Elmer). Various fragments of GPR26 GPCR may be synthesized separately and then combined to produce the full length molecule.

### Biosensors

The GPR26 polypeptides, nucleic acids, probes, antibodies, expression vectors and ligands are useful as (and for the production of) biosensors.

According to Aizawa (1988), Anal. Chem. Symp. 17: 683, a biosensor is defined as being a unique combination of a receptor for molecular recognition, for example a selective layer with immobilized antibodies or receptors such as a GPR26 G-protein coupled receptor, and a transducer for transmitting the values measured. One group of such biosensors will detect the change which is caused in the optical properties of a surface layer due to the interaction of the receptor with the surrounding medium. Among such techniques may be mentioned especially ellipsometry and surface plasmon resonance. Biosensors incorporating GPR26 may be used to detect the presence or level of GPR26 ligands, for example, peptide hormones, nucleotides such as purines or purine analogues, or analogues of these ligands. The construction of such biosensors is well known in the art.

Thus, cell lines expressing GPR26 receptor may be used as reporter systems for detection of ligands via receptor-promoted formation of [3H]inositol phosphates or other second messengers (Watt et al., 1998, J Biol Chem May 29; 273(22):14053-8). Receptor-ligand biosensors are also described in Hoffman et al., 2000, Proc Natl Acad Sci USA October 10; 97(21):11215-20. Optical and other biosensors comprising GPR26 may also be used to detect the level or presence of interaction with G-proteins and other proteins, as described by, for example, Figler et al, 1997, Biochemistry December 23; 36(51):16288-99 and Sarrio et al., 2000, Mol Cell Biol 2000 July; 20(14):5164-74). Sensor units for biosensors are described in, for example, U.S. Pat. No. 5,492,840.

### Screening Assays

The GPR26 GPCR polypeptide of the present invention, may be employed in a screening process for agents which bind the receptor and which activate (agonists) or inhibit activation of (antagonists) of GPR26. A method of identifying an agent suitable for therapy of mental illness comprises the step of determining whether a candidate agent affects the activity of GPR26. As used herein, the term “agent” refers to a moiety that could affect the activity of a receptor, as will be appreciated by one skilled in the art. Preferably, the agent is a compound. A candidate agent is therefore a substance that is tested to determine whether it (the candidate agent) affects the activity of GPR26.

The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. Preferably the compound is an antibody.

Methods of determining whether a candidate agent binds to or otherwise affects the activity of a receptor (GPR26) are well known in the art. In vitro, in vivo and in silico (i.e. the use of a docking algorithm) methods are within the scope of the invention.

Polypeptides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1(2): Chapter 5 (1991).

In general, GPR26 GPCR agonists and antagonists are employed for therapeutic and prophylactic purposes for such mental illnesses, as defined above.

Rational design of candidate compounds likely to be able to interact with GPR26 GPCR protein may be based upon structural studies of the conformation of a polypeptide according to the invention. Preferred structural analyses include x-ray crystallography and NMR. These provide guidance as to which amino acid residues form molecular contact regions. For a detailed description of protein structural determination, see, e.g., Blundell and Johnson (1976) Protein Crystallography, Academic Press, New York.

An alternative to rational design uses a screening procedure which involves in general producing appropriate cells which express the GPR26 receptor polypeptide of the present invention on the surface thereof. Such cells include cells from animals, yeast, Drosophila or E coli. Cells expressing the receptor (or cell membrane containing the expressed receptor) are then contacted with a test compound to observe binding, stimulation or inhibition of a functional response. For example, Xenopus two electrodes voltage clamp system (Wagner et al, Supra) may be used. Furthermore, microphysiometric assays may be employed to assay GPR26 receptor activity. Activation of a wide variety of secondary messenger systems results in extrusion of small amounts of acid from a cell. The acid formed is largely as a result of the increased metabolic activity required to fuel the intracellular signalling process. The pH changes in the media surrounding the cell are very small but are detectable by, for example, the cytosensor microphysiometer (Molecular Devices Ltd., Menlo Park, Calif.). The cytosensor is thus capable of detecting the activation of a receptor which is coupled to an energy utilizing intracellular signaling pathway such as the G-protein coupled receptor of the present invention.

Instead of testing each candidate compound individually with the GPR26 receptor, a library or bank of candidate ligands may advantageously be produced and screened. Thus, for example, a bank of over 200 putative receptor ligands has been assembled for screening. The bank comprises: transmitters, hormones and chemokines known to act via a human 7TM receptor; naturally occurring compounds which may be putative agonists for a human 7TM receptor; non-mammalian, biologically active peptides for which a mammalian counterpart has not yet been identified; and compounds not found in nature, but which activate 7TM receptors with unknown natural ligands. This bank is used to screen the receptor for known ligands, using both functional (i.e. calcium, cAMP, microphysiometer, oocyte electrophysiology, etc, see elsewhere) as well as binding assays as described in further detail elsewhere. However, a large number of mammalian receptors exist for which there remains, as yet, no agonist or antagonist. Thus, active ligands for these receptors may not be included within the ligands banks as identified to date. Accordingly, the GPR26 receptor of the invention is also functionally screened (using calcium, cAMP, microphysiometer, oocyte electrophysiology, etc., functional screens) against tissue extracts to identify natural ligands. Extracts that produce positive functional responses can be sequentially subfractionated, with the fractions being assayed as described here, until an activating ligand is isolated and identified.

7TM receptors which are expressed in HEK 293 cells have been shown to be coupled functionally to activation of PLC and calcium mobilization and/or cAMP stimulation or inhibition. One screening technique therefore includes the use of cells which express the GPR26 GPCR receptor of this invention (for example, transfected Xenopus oocytes, CHO or HEK293 cells) in a system which measures extracellular pH or intracellular calcium changes caused by receptor activation. In this technique, compounds may be contacted with cells expressing the receptor polypeptide of the present invention. A second messenger response, e.g., signal transduction, pH changes, or changes in calcium level, is then measured to determine whether the potential compound activates or inhibits the receptor.

Another method involves screening for receptor inhibitors by determining inhibition or stimulation of GPR26 receptor-mediated cAMP and/or adenylate cyclase accumulation. Such a method involves transfecting a eukaryotic cell with the receptor of this invention to express the receptor on the cell surface. The cell is then exposed to potential antagonists in the presence of the receptor of this invention. The amount of cAMP accumulation is then measured. If the potential antagonist binds the receptor, and thus inhibits receptor binding, the levels of receptor-mediated cAMP, or adenylate cyclase, activity will be reduced or increased.

Another method for detecting agonists or antagonists for the receptor of the present invention is the yeast based technology as described in U.S. Pat. No. 5,482,835, incorporated by reference herein.

Where the candidate compounds are proteins, in particular antibodies or peptides, libraries of candidate compounds may be screened using phage display techniques. Phage display is a protocol of molecular screening which utilises recombinant bacteriophage. The technology involves transforming bacteriophage with a gene that encodes one compound from the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of binding to a polypeptide or peptide of the invention are enriched by selection strategies based on affinity interaction. The successful candidate agents are then characterised. Phage display has advantages over standard affinity ligand screening technologies. The phage surface displays the candidate agent in a three-dimensional configuration, more closely resembling its naturally occurring conformation. This allows for more specific and higher affinity binding for screening purposes.

Another method of screening a library of compounds utilises eukaryotic or prokaryotic host cells which are stably transformed with recombinant DNA molecules expressing a library of compounds. Such cells, either in viable or fixed form, can be used for standard binding-partner assays. See also Parce et al. (1989) Science 246:243-247; and Owicki et al. (1990) Proc. Nat'l Acad. Sci. USA 87; 4007-4011, which describe sensitive methods to detect cellular responses. Competitive assays are particularly useful, where the cells expressing the library of compounds are contacted or incubated with a labelled antibody known to bind to a GPR26 polypeptide of the present invention, such as ¹²⁵I-antibody, and a test sample such as a candidate compound whose binding affinity to the binding composition is being measured. The bound and free labelled binding partners for the polypeptide are then separated to assess the degree of binding. The amount of test sample bound is inversely proportional to the amount of labelled antibody binding to the polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilized, unpurified or solubilized purified polypeptide or peptides, for example extracted from transformed eukaryotic or prokaryotic host cells. This allows for a “molecular” binding assay with the advantages of increased specificity, the ability to automate, and high drug test throughput.

Another technique for candidate compound screening involves an approach which provides high throughput screening for new compounds having suitable binding affinity, e.g., to a polypeptide of the invention, and is described in detail in WO84/03564. First, large numbers of different small peptide test compounds are synthesized on a solid substrate, e.g., plastic pins or some other appropriate surface; see Fodor et al. (1991). Then all the pins are reacted with solubilized polypeptide of the invention and washed. The next step involves detecting bound polypeptide. Compounds which interact specifically with the polypeptide will thus be identified.

Ligand-binding assays provide a direct method for ascertaining receptor pharmacology and are adaptable to a high throughput format. The purified ligand for a receptor may be radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

The assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed.

Further, the assays may simply comprise the steps of mixing a candidate compound with a solution containing a GPR26 GPCR polypeptide to form a mixture, measuring GPR26 GPCR activity in the mixture, and comparing the GPR26 GPCR activity of the mixture to a standard.

The GPR26 GPCR cDNA, protein and antibodies to the protein may also be used to configure assays for detecting the effect of added compounds on the production of GPR26 GPCR mRNA and protein in cells. For example, an ELISA may be constructed for measuring secreted or cell associated levels of GPR26 GPCR protein using monoclonal and polyclonal antibodies by standard methods known in the art, and this can be used to discover agents which may inhibit or enhance the production of GPR26 GPCR (also called antagonist or agonist, respectively) from suitably manipulated cells or tissues. Standard methods for conducting screening assays are well understood in the art.

Examples of potential GPR26 GPCR antagonists include antibodies or, in some cases, nucleotides and their analogues, including purines and purine analogues, oligonucleotides or proteins which are closely related to the ligand of the GPR26 GPCR, e.g., a fragment of the ligand, or small molecules which bind to the receptor but do not elicit a response, so that the activity of the receptor is prevented. The present invention therefore also provides a compound capable of binding specifically to a GPR26 polypeptide and/or peptide of the present invention.

The materials necessary for such screening to be conducted may be packaged into a screening kit. Such a screening kit is useful for identifying agonists, antagonists, ligands, receptors, substrates, enzymes, etc. for GPR26 GPCR polypeptides or compounds which decrease or enhance the production of GPR26 GPCR polypeptides. The screening kit comprises: (a) a GPR26 GPCR polypeptide; (b) a recombinant cell expressing a GPR26 GPCR polypeptide; (c) a cell membrane expressing a GPR26 GPCR polypeptide; or (d) an antibody to a GPR26 GPCR polypeptide. The screening kit may optionally comprise instructions for use.

### Transgenic Animals

The present invention further encompasses transgenic animals capable of expressing natural or recombinant GPR26 GPCR, or a homologue, variant or derivative, at elevated or reduced levels compared to the normal expression level. Included are transgenic animals (“GPR26 knockouts”) which do not express functional GPR26 receptor as a result of one or more loss of function mutations, including a deletion (of both alleles i.e. −/−), of the GPR26 gene. These animals are said to have a functionally-disrupted endogenous GPR26 gene. A non-human animal that does not express any functional (GPR26) polypeptide is often referred to as having a “null” mutation.

Preferably, the transgenic animal is a non-human mammal, such as a pig, a sheep or a rodent. Most preferably the transgenic animal is a mouse or a rat. Such transgenic animals may be used in screening procedures to identify agonists and/or antagonists of GPR26 GPCR, as well as to test for their efficacy as treatments for diseases in vivo.

For example, transgenic animals that have been engineered to be deficient in the production of GPR26 GPCR may be used in assays to identify agonists and/or antagonists of GPR26 GPCR. One assay is designed to evaluate a potential drug (aa candidate ligand or compound) to determine if it produces a physiological response in the absence of GPR26 GPCR receptors. This may be accomplished by administering the drug to a transgenic animal as discussed above, and then assaying the animal for a particular response, which will be apparent to the skilled person. Tissues derived from the GPR26 knockout animals may be used in receptor binding assays to determine whether the potential drug (a candidate ligand or compound) binds to the GPR26 receptor. Such assays can be conducted by obtaining a first receptor preparation from the transgenic animal engineered to be deficient in GPR26 receptor production and a second receptor preparation from a source known to bind any identified GPR26 ligands or compounds. In general, the first and second receptor preparations will be similar in all respects except for the source from which they are obtained. For example, if brain tissue from a transgenic animal (such as described above and below) is used in an assay, comparable brain tissue from a normal (wild type) animal is used as the source of the second receptor preparation. Each of the receptor preparations is incubated with a ligand known to bind to GPR26 receptors, both alone and in the presence of the candidate ligand or compound. Preferably, the candidate ligand or compound will be examined at several different concentrations.

The extent to which binding by the known ligand is displaced by the test compound is determined for both the first and second receptor preparations. Tissues derived from transgenic animals may be used in assays directly or the tissues may be processed to isolate membranes or membrane proteins, which are themselves used in the assays. A preferred transgenic animal is the mouse. The ligand may be labeled using any means compatible with binding assays. This would include, without limitation, radioactive, enzymatic, fluorescent or chemiluminescent labeling (as well as other labelling techniques as described in further detail above).

Furthermore, antagonists of GPR26 GPCR receptor may be identified by administering candidate compounds, etc, to wild type animals expressing functional GPR26, and animals identified which exhibit any of the phenotypic characteristics associated with reduced or abolished expression of GPR26 receptor function.

Methods for generating a non-human transgenic animal are well known in the art and any suitable method may be used. In an exemplary embodiment, the transgenic non-human animals of the invention are produced by introducing transgenes into the germline of the non-human animal. One or several copies of the construct may be incorporated into the genome. Embryonal target cells at various developmental stages can be used to introduce transgenes. Different methods are used depending on the stage of development of the embryonal target cell. Introduction of the transgene into the embryo can be accomplished by any means known in the art such as, for example, microinjection, electroporation, or lipofection. Following introduction of the transgene construct into the fertilized egg, the egg may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a GPR26 GPCR receptor. Further, in such embodiments the sequence will be attached to a transcriptional control element, e.g., a promoter, which preferably allows the expression of the transgene product in a specific type of cell.

An alternative target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

We also provide for non-human transgenic animals, where the transgenic animal is characterized by having an altered GPR26 gene, preferably as described above, as models for GPR26 receptor function. Alterations to the gene include deletions or other loss of function mutations, introduction of an exogenous gene having a nucleotide sequence with targeted or random mutations, introduction of an exogenous gene from another species, or a combination thereof. The transgenic animals may be either homozygous or heterozygous for the alteration. The animals and cells derived therefrom are useful for screening biologically active agents that may modulate GPR26 receptor function. The screening methods are of particular use for determining the specificity and action of potential therapies for mental illness. The animals are useful as a model to investigate the role of GPR26 receptors in normal brain function.

Another aspect of the invention pertains to a transgenic nonhuman animal having a functionally disrupted endogenous GPR26 gene but which also carries in its genome, and expresses, a transgene encoding a heterologous GPR26 protein (i.e., a GPR26 from another species). Preferably, the animal is a mouse and the heterologous GPR26 is a human GPR26. An animal, or cell lines derived from such an animal of the invention, which has been reconstituted with human GPR26, can be used to identify agents that inhibit human GPR26 in vivo and in vitro. For example, a stimulus that induces signalling through human GPR26 can be administered to the animal, or cell line, in the presence and absence of an agent to be tested and the response in the animal, or cell line, can be measured. An agent that inhibits human GPR26 in vivo or in vitro can be identified based upon a decreased response in the presence of the agent compared to the response in the absence of the agent.

The present invention also provides for a GPR26 GPCR-deficient transgenic non-human animal (a “GPR26 GPCR knock-out”). Such an animal is one which expresses lowered or no GPR26 GPCR activity, preferably as a result of an endogenous GPR26 GPCR genomic sequence being disrupted or deleted. Preferably, such an animal expresses no GPCR activity. More preferably, the animal expresses no activity of the GPR26 GPCR shown as SEQ ID NO: 3 or SEQ ID NO: 5. GPR26 GPCR knock-outs may be generated by various means known in the art, as described in further detail below.

The present invention also pertains to a nucleic acid construct for functionally disrupting a GPR26 gene in a host cell. The nucleic acid construct comprises: a) a non-homologous replacement portion; b) a first homology region located upstream of the non-homologous replacement portion, the first homology region having a nucleotide sequence with substantial identity to a first GPR26 gene sequence; and c) a second homology region located downstream of the non-homologous replacement portion, the second homology region having a nucleotide sequence with substantial identity to a second GPR26 gene sequence, the second GPR26 gene sequence having a location downstream of the first GPR26 gene sequence in a naturally occurring endogenous GPR26 gene. Additionally, the first and second homology regions are of sufficient length for homologous recombination between the nucleic acid construct and an endogenous GPR26 gene in a host cell when the nucleic acid molecule is introduced into the host cell. In a preferred embodiment, the non-homologous replacement portion comprises an expression reporter, preferably including lacZ and a positive selection expression cassette, preferably including a neomycin phosphotransferase gene operatively linked to a regulatory element(s).

Preferably, the first and second GPR26 gene sequences are derived from SEQ ID No. 6, and may include SEQ ID NO: 4, or a homologue, variant or derivative thereof.

Another aspect of the invention pertains to recombinant vectors into which the nucleic acid construct of the invention has been incorporated. Yet another aspect of the invention pertains to host cells into which the nucleic acid construct of the invention has been introduced to thereby allow homologous recombination between the nucleic acid construct and an endogenous GPR26 gene of the host cell, resulting in functional disruption of the endogenous GPR26 gene. The host cell can be a mammalian cell that normally expresses GPR26 from the brain, or a pluripotent cell, such as a mouse embryonic stem cell. Further development of an embryonic stem cell into which the nucleic acid construct has been introduced and homologously recombined with the endogenous GPR26 gene produces a transgenic nonhuman animal having cells that are descendant from the embryonic stem cell and thus carry the GPR26 gene disruption in their genome. Animals that carry the GPR26 gene disruption in their germline can then be selected and bred to produce animals having the GPR26 gene disruption in all somatic and germ cells. Such mice can then be bred to homozygosity for the GPR26 gene disruption.

A GPR26 GPCR deficient transgenic animal may be generated as described in Examples 1 and 2.

### Antibodies

For the purposes of this invention, the term “antibody”, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. The antibodies and fragments thereof may be humanised antibodies, for example as described in EP-A-0239400. Furthermore, antibodies with fully human variable regions (or their fragments), for example, as described in U.S. Pat. Nos. 5,545,807 and 6,075,181 may also be used. Neutralizing antibodies, i.e., those which inhibit biological activity of the substance amino acid sequences, are especially preferred for diagnostics and therapeutics.

Antibodies may be produced by standard techniques, such as by immunisation or by using a phage display library.

A polypeptide or peptide of the present invention may be used to develop an antibody by known techniques. Such an antibody may be capable of binding specifically to the GPR26 GPCR protein or homologue, fragment, etc.

If polyclonal antibodies are desired, a selected mammal (e.g., mouse, rabbit, goat, horse, etc.) may be immunised with an immunogenic composition comprising a polypeptide or peptide of the present invention. Depending on the host species, various adjuvants, including Freunds Adjuvants and Aluminium Hydroxid, may be used to increase immunological response. BCG (Bacilli Calmette-Guerin) and Corynebacterium parvum are potentially useful human adjuvants which may be employed if purified the substance amino acid sequence is administered to immunologically compromised individuals for the purpose of stimulating systemic defence.

Monoclonal antibodies directed against epitopes obtainable from a polypeptide or peptide of the present invention can also be readily produced by one skilled in the art. The general methodology for making monoclonal antibodies by hybridomas is well known.

In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from a polypeptide or peptide of the present invention are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

The above-described antibodies may be employed to isolate or to identify clones expressing the polypeptide or to purify the polypeptides by affinity chromatography.

Antibodies against GPR26 GPCR polypeptides may also be employed to treat mental illness.

### Diagnostic Assays

GPR26 is implicated in mental illness. Therefore, detecting the presence and/or activity of GPR26 in an individual, or preferably in a sample isolated from an individual, allows a diagnosis to be made on the presence of or susceptibility to mental illness, as will be appreciated by one skilled in the art.

This invention also relates to the use of GPR26 GPCR polynucleotides and polypeptides (as well as homologues, variants and derivatives thereof) for use in diagnosis as diagnostic reagents or in genetic analysis. Nucleic acids complementary to or capable of hybridising to GPR26 GPCR nucleic acids (including homologues, variants and derivatives), as well as antibodies against GPR26 polypeptides are also useful in such assays.

In a prepared embodiment, the diagnosis is performed in vitro, i.e. it is not performed on the body of a person or animal that is being diagnosed.

A method of determining the presence or susceptibility of mental illness in an individual (i.e. a patient) comprises the steps of (i) determining the expression level of GPR26 in a sample isolated from the individual, and (i) determining, compared to a control, whether the individual from which the sample was isolated has, or is susceptible to mental illness.

Detection of a mutated form of the GPR26 GPCR gene associated with a dysfunction will provide a diagnostic tool that can add to or define a diagnosis of a disease or susceptibility to a disease which results from under-expression, over-expression or altered expression of GPR26 GPCR. Individuals carrying mutations in the GPR26 GPCR gene (including control sequences) may be detected at the DNA level by a variety of techniques that are known in the art. Preferably, the region of interest (comprising GPR26 GPCR) is amplified using PCR, prior to mutation analysis by direct sequencing or other commonly used methods.

For example, DNA may be isolated from a patient and the DNA polymorphism pattern of GPR26 determined. The identified pattern is compared to controls of patients known to be suffering from a disease associated with over-, under- or abnormal expression of GPR26. Patients expressing a genetic polymorphism pattern associated with GPR26 associated disease may then be identified. Genetic analysis of the GPR26 GPCR gene may be conducted by any technique known in the art. For example, individuals may be screened by determining DNA sequence of a GPR26 allele, by RFLP (Restriction Fragment Length Polymorphism) or SSCP (Single Strand Conformation Polymorphism) analysis. Patients may be identified as having a genetic predisposition for a disease associated with the over-, under-, or abnormal expression of GPR26 by detecting the presence of a DNA polymorphism in the gene sequence for GPR26 or any sequence controlling its expression.

Patients so identified can then be treated to prevent the occurrence of GPR26 associated disease, or more aggressively in the early stages of GPR26 associated disease to prevent the further occurrence or development of the disease. GPR26 associated diseases are mental illnesses.

The present invention further discloses a kit for the identification of a patient's genetic polymorphism pattern associated with GPR26 associated disease. The kit includes DNA sample collecting means and means for determining a genetic polymorphism pattern, which is then compared to control samples to determine a patient's susceptibility to GPR26 associated disease. Kits for diagnosis of a GPR26 associated disease comprising GPR26 polypeptide and/or an antibody against such a polypeptide (or fragment of it) are also provided.

The diagnostic assays offer a process for diagnosing or determining a susceptibility to mental illness, through detection of mutation in the GPR26 GPCR gene by the methods described.

The presence of GPR26 GPCR polypeptides and nucleic acids may be detected in a sample. Thus, infections and diseases as listed above can be diagnosed by methods comprising determining from a sample derived from a subject an abnormally decreased or increased level of the GPR26 GPCR polypeptide or GPR26 GPCR mRNA. The sample may comprise a cell or tissue sample from an organism suffering or suspected to be suffering from a disease associated with increased, reduced or otherwise abnormal GPR26 GPCR expression, including spatial or temporal changes in level or pattern of expression. The level or pattern of expression of GPR26 in an organism suffering from or suspected to be suffering from such a disease may be usefully compared with the level or pattern of expression in a normal organism as a means of diagnosis of disease.

In general therefore, the invention includes a method of detecting the presence of a nucleic acid comprising a GPR26 GPCR nucleic acid in a sample, by contacting the sample with at least one nucleic acid probe which is specific for said nucleic acid and monitoring said sample for the presence of the nucleic acid. For example, the nucleic acid probe may specifically bind to the GPR26 GPCR nucleic acid, or a portion of it, and binding between the two detected; the presence of the complex itself may also be detected. Furthermore, the invention encompasses a method of detecting the presence of a GPR26 GPCR polypeptide by contacting a cell sample with an antibody capable of binding the polypeptide and monitoring said sample for the presence of the polypeptide. This may conveniently be achieved by monitoring the presence of a complex formed between the antibody and the polypeptide, or monitoring the binding between the polypeptide and the antibody. Methods of detecting binding between two entities are known in the art, and include FRET (fluorescence resonance energy transfer), surface plasmon resonance, etc.

Decreased or increased expression can be measured at the RNA level using any of the methods well known in the art for the quantitation of polynucleotides, such as, for example, PCR, RT-PCR, RNase protection, Northern blotting and other hybridization methods. Assay techniques that can be used to determine levels of a protein, such as a GPR26 GPCR, in a sample derived from a host are well-known to those of skill in the art. Such assay methods include radioimmunoassays, competitive-binding assays, Western Blot analysis and ELISA assays.

The present invention relates to a diagnostic kit for a disease or susceptibility to mental illness.

The diagnostic kit comprises a GPR26 GPCR polynucleotide or a fragment thereof; a complementary nucleotide sequence; a GPR26 GPCR polypeptide or a fragment thereof, or an antibody to a GPR26 GPCR polypeptide.

### Chromosome Assays

The nucleotide sequences of the present invention are also valuable for chromosome identification. The sequence is specifically targeted to and can hybridize with a particular location on an individual human chromosome. As described above, human GPR26 GPCR is found to map to chromosomal location 10q26.2-q26.3.

The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found, for example, in V. McKusick, Mendelian inheritance in Man (available on line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

### Prophylactic and Therapeutic Methods

This invention provides methods of treating an abnormal condition related to both an excess of and insufficient amount of GPR26 GPCR activity.

If the activity of GPR26 GPCR is in excess, several approaches are available. One approach comprises administering to a subject an inhibitor compound (antagonist) as hereinabove described along with a pharmaceutically acceptable carrier in an amount effective to inhibit activation by blocking binding of ligands to the GPR26 GPCR, or by inhibiting a second signal, and thereby alleviating the abnormal condition.

In another approach, soluble forms of GPR26 GPCR polypeptides still capable of binding the ligand in competition with endogenous GPR26 GPCR may be administered. Typical embodiments of such competitors comprise fragments of the GPR26 GPCR polypeptide.

In still another approach, expression of the gene encoding endogenous GPR26 GPCR can be inhibited using expression blocking techniques. Known such techniques involve the use of antisense sequences, either internally generated or separately administered. See, for example, O'Connor, J Neurochem (1991) 56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al., Nucleic Acids Res (1979) 6:3073; Cooney et al., Science (1988) 241:456; Dervan et al., Science (1991) 251:1360. These oligomers can be administered per se or the relevant oligomers can be expressed in vivo.

For treating abnormal conditions related to an under-expression of GPR26 GPCR and its activity, several approaches are also available. One approach comprises administering to a subject a therapeutically effective amount of a compound which activates GPR26 GPCR, i.e., an agonist as described above, in combination with a pharmaceutically acceptable carrier, to thereby alleviate the abnormal condition. Alternatively, gene therapy may be employed to effect the endogenous production of GPR26 GPCR by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

### Formulation and Administration

Peptides, such as the soluble form of GPR26 GPCR polypeptides, and agonists and antagonist peptides or small molecules, may be formulated in combination with a suitable pharmaceutical carrier. Such formulations comprise a therapeutically effective amount of the polypeptide or compound, and a pharmaceutically acceptable carrier or excipient. Such carriers include but are not limited to, saline, buffered saline, dextrose, water, glycerol, ethanol, and combinations thereof. Formulation should suit the mode of administration, and is well within the skill of the art. The invention further relates to pharmaceutical packs and kits comprising one or more containers filled with one or more of the ingredients of the aforementioned compositions of the invention.

Polypeptides and other compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localized, in the form of salves, pastes, gels and the like.

The dosage range required depends on the choice of peptide, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

### Pharmaceutical Compositions

The present invention provides a pharmaceutical composition comprising administering a therapeutically effective amount of the combination of polypeptides, peptides, or antibody of the present invention and optionally a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

### Vaccines

Another embodiment of the invention relates to a method for inducing an immunological response in a mammal which comprises inoculating the mammal with the GPR26 GPCR polypeptide, or a fragment thereof, adequate to produce antibody and/or T cell immune response to protect said animal from mental illness.

Yet another embodiment of the invention relates to a method of inducing immunological response in a mammal which comprises delivering a GPR26 GPCR polypeptide via a vector directing expression of a GPR26 GPCR polynucleotide in vivo in order to induce such an immunological response to produce antibody to protect said animal from diseases.

A further embodiment of the invention relates to an immunological/vaccine formulation (composition) which, when introduced into a mammalian host, induces an immunological response in that mammal to a GPR26 GPCR polypeptide wherein the composition comprises a GPR26 GPCR polypeptide or GPR26 GPCR gene. The vaccine formulation may further comprise a suitable carrier.

Since the GPR26 GPCR polypeptide may be broken down in the stomach, it is preferably administered parenterally (including subcutaneous, intramuscular, intravenous, intradermal etc. injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Vaccines may be prepared from two or more polypeptides or peptides of the present invention.

The preparation of vaccines which contain immunogenic polypeptides or peptide(s) as active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the protein encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further examples of adjuvants and other agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

Typically, adjuvants such as Amphigen (oil-in-water), Alhydrogel (aluminum hydroxide), or a mixture of Amphigen and Alhydrogel are used. Only aluminum hydroxide is approved for human use.

The proportion of immunogen and adjuvant can be varied over a broad range so long as both are present in effective amounts. For example, aluminum hydroxide can be present in an amount of about 0.5% of the vaccine mixture (Al₂O₃ basis). Conveniently, the vaccines are formulated to contain a final concentration of immunogen in the range of from 0.2 to 200 μg/ml, preferably 5 to 50 μg/ml, most preferably 15 μg/ml.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer

The polypeptides of the invention may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The pharmaceutical and vaccine compositions of the present invention may be administered by direct injection. The composition may be formulated for parenteral, mucosal, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestible solution; a parenteral route where delivery is by an ingestible form, such as, for example, an intravenous, intramuscular or subcutaneous route.

The term “co-administered” means that the site and time of administration of each of for example, the polypeptide of the present invention and an additional entity such as adjuvant are such that the necessary modulation of the immune system is achieved. Thus, whilst the polypeptide and the adjuvant may be administered at the same moment in time and at the same site, there may be advantages in administering the polypeptide at a different time and to a different site from the adjuvant. The polypeptide and adjuvant may even be delivered in the same delivery vehicle—and the polypeptide and the antigen may be coupled and/or uncoupled and/or genetically coupled and/or uncoupled.

The polypeptide, polynucleotide, peptide, nucleotide, antibody of the invention and optionally an adjuvant may be administered separately or co-administered to the host subject as a single dose or in multiple doses.

The vaccine composition and pharmaceutical compositions of the present invention may be administered by a number of different routes such as injection (which includes parenteral, subcutaneous and intramuscular injection) intranasal, mucosal, oral, intra-vaginal, urethral or ocular administration.

The vaccines and pharmaceutical compositions of the present invention may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, may be 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

The invention is further described by the following non-limiting examples.

### EXAMPLES

### Example 1

### Transgenic GPR26 Knock-Out Mouse: Construction of GPR26 Gene Targeting Vector

A PAC containing the GPR26 gene was identified from a murine PAC library using a radioactively labelled probe derived from a section of the coding sequence. A 79 kb gapped murine genomic contig was assembled using an in-house restriction site anchored PCR method similar to GeneWalker (Clontech). Further bio-informatic work increased the contig size to 182 kb. This contig provided sufficient flanking sequence information to enable the design of homologous arms to clone into the targeting vector.

The murine GPR26 gene has 3 coding exons. The targeting strategy is designed to remove the majority of the first coding exon, including 578 bases of the 7tm encoding region. A 1.6 kb 5′ homologous arm and a 4.0 kb 3′ homologous arm flanking the region to be deleted are amplified by PCR and the fragments are cloned into the targeting vector. The 5′ end of each oligonucleotide primer used to amplify the arms is synthesised to contain a different recognition site for a rare-cutting restriction enzyme, compatible with the cloning sites of the vector polylinkers situated at either end of the reporter/selection cassette, and absent from the arms themselves. In the case of GPR26, the primers are designed as listed in the primer table below, with 5′ arm cloning sites of NotI/SpeI and 3′arm cloning sites of AscI/FseI.

In addition to the arm primer pairs (5′armF/5′armR) and (3′armF/3′armR), further primers specific to the GPR26 locus are designed for the following purposes: 5′ and 3′ probe primer pairs (5′prF/5′prR and 3′prF/3′prR) to amplify two short 150-300 bp fragments of non-repetitive genomic DNA external to and extending beyond each arm, to allow Southern analysis of the targeted locus, in isolated putative targeted clones; a mouse genotyping primer pair (hetF and hetR) which allows differentiation between wild-type, heterozygote and homozygous mice, when used in a multiplex PCR with a vector specific primer, in this case, asc350; and lastly, a target screening primer (5′scr) which anneals upstream of the end of the 5′ arm region, and which produces a target event specific 1.7 kb amplimer when paired with a primer specific to the 5′ end of the vector, in this case DR1. This amplimer can only be derived from template DNA from cells where the desired genomic alteration has occurred and allows the identification of correctly targeted cells from the background of clones containing randomly integrated copies of the vector. The location of these primers and the genomic structure of the regions of the GPR26 locus used in the targeting strategy is shown in FIG. 6 (and SEQ ID NO. 6).

| GPR26 Primer Sequences (SEQ ID Nos. 7 to 19, respectively) | | |
|---|---|---|
| mus2Maugh 5′prF | (SEQ ID No. 7) | TGGTGCTTAGCATCTGTCAAGGTGTAG |
| mus2Maugh 5′prR | (SEQ ID No. 8) | CTACACTGTATTTCAGAGCCTGGTGAG |
| mus2Maugh 5′scr | (SEQ ID No. 9) | AGGCTCTGAAATACAGTGTAGGTGGTC |
| mus2Maugh 5′armF | (SEQ ID No. 10) | aaagcggccgcGGAGGTAGCATCCCATAGAAAGGCAAG |
| mus2Maugh 5′armR | (SEQ ID No. 11) | tttactagTTGGACAGCAGCGACACGCCGATAGTG |
| mus2Maugh 3′armF | (SEQ ID No. 12) | aaaggcgcgccTGTTGGTGGACATACACCCCAGGTGAG |
| mus2Maugh 3′armR | (SEQ ID No. 13) | tttggccggccGGTCCCAGTAACAGCAGCTCCTGAGTC |
| mus2Maugh 3′prF | (SEQ ID No. 14) | AGCCAGAGAACCTGCCTGCTTGGTCTC |
| mus2Maugh 3′prR | (SEQ ID No. 15) | ACTCGCAGATGAGGACACTGGGAACAG |
| mus2Maugh het | (SEQ ID No. 16) | GCTCGGCTTCCACCAGCTATATGCCTC |
| mus2Maugh hetR a350 | (SEQ ID No. 17) | ACTCACCTGGGGTGTATGTCCACCAAC |
| Asc350 | (SEQ ID No. 18) | GTCGTGACCCATGGCGATGCCTGCTTG |
| DR1 | (SEQ ID No. 19) | CATGCCGCCTGCGCCCTATTGATCATG |

The position of the homology arms is chosen to functionally disrupt the GPR26 gene. A targeting vector is prepared where the GPR26 region to be deleted is replaced with non-homologous sequences composed of an endogenous gene expression reporter (a frame independent lacZ gene) upstream of a selection cassette composed of a promoted neomycin phosphotransferase (neo) gene arranged in the same orientation as the GPR26 gene.

Once the 5′ and 3′ homology arms have been cloned into the targeting vector, a large highly pure DNA preparation is made using standard molecular biology techniques. 20 μg of the freshly prepared endotoxin-free DNA is restricted with another rare-cutting restriction enzyme SwaI, present at a unique site in the vector backbone between the ampicillin resistance gene and the bacterial origin of replication. The linearized DNA is then precipitated and resuspended in 100 μl of Phosphate Buffered Saline, ready for electroporation.

24 hours following electroporation the transfected cells are cultured for 9 days in medium containing 200 μg/ml neomycin. Clones are picked into 96 well plates, replicated and expanded before being screened by PCR (using primers 5′scr and DR1, as described above) to identify clones in which homologous recombination has occurred between the endogenous GPR26 gene and the targeting construct. Positive clones can be identified at a rate of 1 to 5%. These clones are expanded to allow replicas to be frozen and sufficient high quality DNA to be prepared for Southern blot confirmation of the targeting event using the external 5′ and 3′ probes prepared as described above, all using standard procedures (Russ et al, Nature 2000 Mar. 2; 404(6773):95-99). When Southern blots of DNA digested with diagnostic restriction enzymes are hybridized with an external probe, homologously targeted ES cell clones are verified by the presence of a mutant band as well an unaltered wild-type band. For instance, using the 5′ probe, BamHI digested genomic DNA will give a 8.7 kb wild-type band and a 2.4 kb targeted band; and with the 3′ probe, BamHI cut DNA will give a 8.7 kb wild-type band and a 5.5 kb targeted band.

### Example 2

### Transgenic GPR26 Knock-Out Mouse: Generation of GPR26 Deficient Mice

C57BL/6 female and male mice are mated and blastocysts are isolated at 3.5 days of gestation. 10-12 cells from a chosen clone are injected per blastocyst and 7-8 blastocysts are implanted in the uterus of a pseudopregnant F1 female. A litter of chimeric pups are born containing several high level (up to 100%) agouti males (the agouti coat colour indicates the contribution of cells descended from the targeted clone). These male chimeras are mated with female MF1 and 129 mice, and germline transmission is determined by the agouti coat colour and by PCR genotyping respectively.

PCR Genotyping is carried out on lysed tail clips, using the primers hetF and hetR with a third, vector specific primer (asc350). This multiplex PCR allows amplification from the wild-type locus (if present) from primers hetF and hetR giving a 236 bp band. The site for hetF is deleted in the knockout mice, so this amplification will fail from a targeted allele. However, the asc350 primer will amplify a 379 bp band from the targeted locus, in combination with the hetR primer which anneals to a region just inside the 3′ arm. Therefore, this multiplex PCR reveals the genotype of the litters as follows: wild-type samples exhibit a single 236 bp band; heterozygous DNA samples yield two bands at 236 bp and 379 bp; and the homozygous samples will show only the target specific 379 bp band.

### Example 3

### Biological Results

### I) Gene Expression Patterns

### A) RT-PCR

In the human RT-PCR panel the gene is expressed in the Brain only. In the mouse brain RT-PCR panel the gene is expressed broadly in the CNS including the hippocampus, striatum, thalamus, forebrain, cortex, pons and the hypothalamus.

### B) LacZ Staining

Representative tissue slices are made of large organs. Whole small organs and tubes are sliced open, so fixative and stain will penetrate. Tissues are rinsed thoroughly in PBS (phosphate buffered saline) to remove blood or gut contents. Tissues are placed in fixative (PBS containing 2% formaldehyde, 0.2% glutaraldehyde, 0.02% NP40, 1 mM MgCl2, Sodium deoxycholate 0.23 mM) for 30-45 minutes. Following three 5 minute washes in PBS, tissues are placed in Xgal staining solution (4 mM K Ferrocyanide, 4 mMKFerricyanide, 2 mM MgCl2, 1 mg/mIX-gal in PBS) for 18 hours at 30 C. Tissues are PBS washed 3 times, postfixed for 24 hours in 4% formaldehyde, PBS washed again before storage in 70% ethanol.

GPR26 was found to be expressed in the cerebral cortex, hippocampus in particular the CA3 region, amygdala, olfactory cortex, hypothalamus, the lateral septum and the ventral tegmental area. This expression pattern suggests importance in psychiatric disease, cognition and memory.

### II) Behaviour

All animals were housed with free access to food and water under a light-dark cycle of 12 h light/12 h darkness with lights on at 7 am. Animals were tested at set times to avoid circadian effects.

### A) POPMAP Test

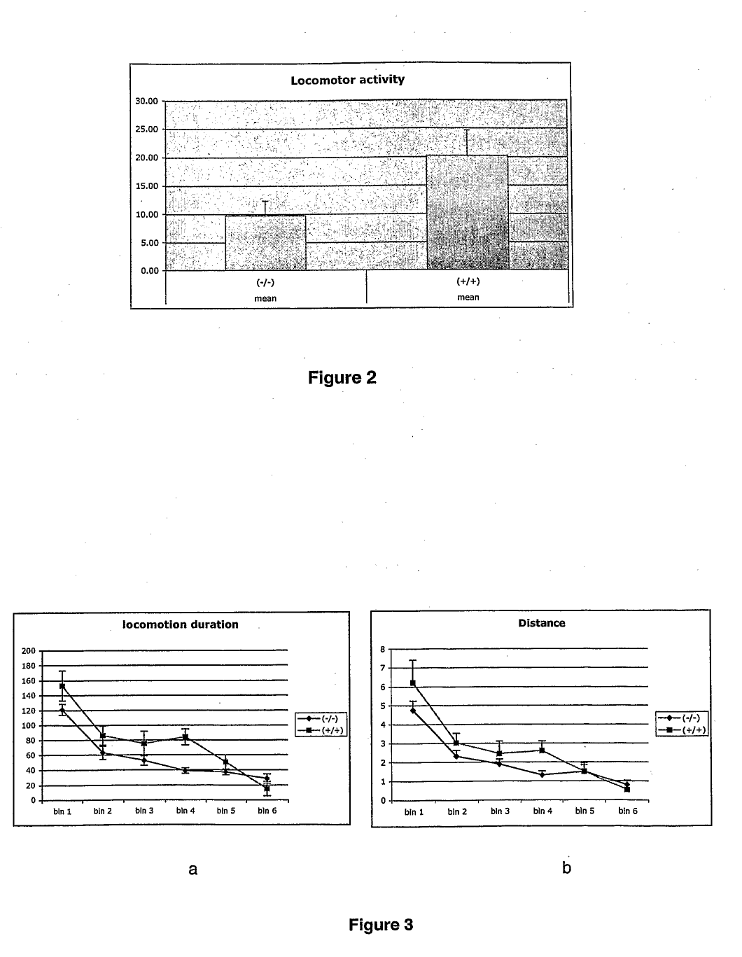

This test is a modified Irwin observational screen and as such will be well known to one skilled in the art. It was observed that knockout mice displayed a reduced locomotor activity compared to wild type control mice (see FIG. 2).

### B) Laboras

The laboras (Laboratory Animal Behaviour Observation Registration and Analysis System) system is an automated behaviour apparatus commercialised by Metris BV (Netherlands). Laboras automatically records and quantifies behaviours using vibrations patterns. All animals are housed with free access to food and water under a light-dark cycle of 12 h light/12 h darkness with lights on at 7 am. Animals are tested at set times to avoid circadian effects. Mice are placed in this apparatus for an initial period of 1 hour with free access to food and water.)

In the Laboras animals showed a reduced locomotor activity (distance traveled and locomotor duration) with no change in collateral behaviour (climbing and grooming), suggesting that the effect of the mutation observed is not a result of reduced coordination or muscular strength, see FIG. 3a and FIG. 3b.

### C) Rotarod

Motor coordination and balance are measured by performance on the rotarod that accelerates at a constant rate from 4 to 40 rpm over the course of 5 min. Mice are left on the rotarod and allowed to perform the test until they either drop from the rod or until 10 min (600 secs) have passed. The latency to fall is recorded. Each mouse is tested three times in the session with 10 minute rest between trials.

No significant difference was observed between knockout mice and wild type mice in the rotarod test.

### D) Grip Strength

The animal's muscular strength is determined using the Bioseb grip strength apparatus. The apparatus consists of an adjustable trough and a push-pull strain gauge with a triangular brass ring, which is grasped by the animal with its forelimbs the animal is pulled on the tail until the grip is broken. The grip strength is quantified in g.

No significant difference in grip strength was observed between knockout mice and wild type controls.

This data further suggests that the observed reduction in locomotor activity does not seem to be a result of reduced coordination or muscular strength. This data plus the expression pattern suggest that the reduction in locomotor activity is of CNS origin.

### E) Porsolt Forced Swim Test

The Porsolt forced swim test is a widely used model of depression based on the assumption that animals will normally try to escape from an aversive stimulus (deep water). When the aversive stimulus is inescapable, the mouse will eventually stop trying to escape and develop an immobile posture. Early cessation of attempts to escape is considered an analogue of stress-induced depression. Antidepressant drug treatments restore the escape behaviour. Mice are placed in a transparent plastic cylinder (19 cm diameter×30 cm high) filled with water such that the feet cannot touch the bottom of the cylinder and the mice cannot escape from the top. Mice are place in the apparatus for a period of 6 minutes, the first 2 minutes are to allowed the animals to habituate, in the following 4 minutes total cumulative resting time and the latency to the first stop are recorded.

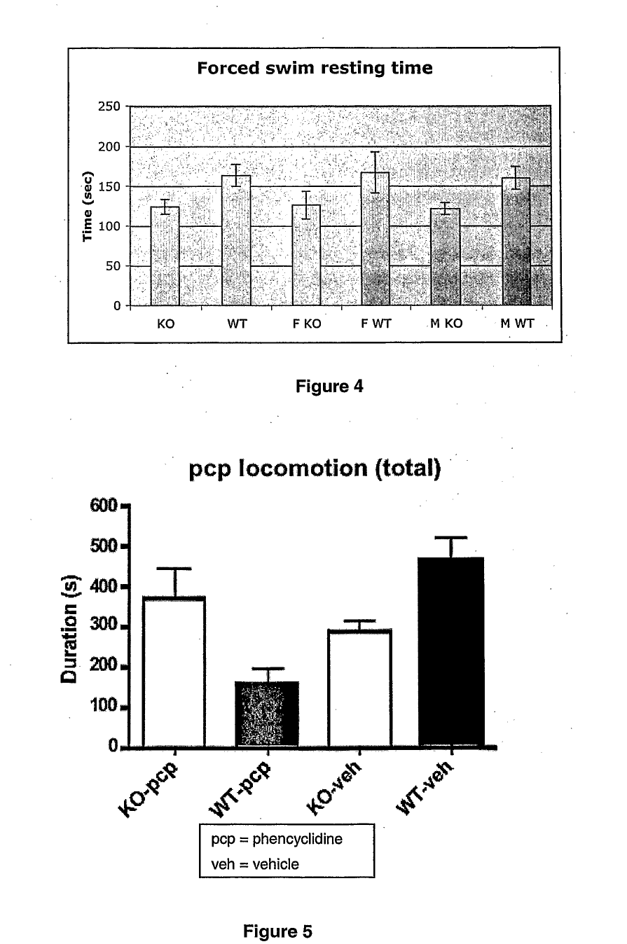

Mutant mice rested less than the wild type controls (wt=163.26±13.9, KO+123.64±9.1, t test P=0.028). This data suggests that GPR26 knockout mice cope better in a stressful environment compared to wild type mice and show less depression (see FIG. 4).

### F) Response to Phencyclidine

Animals were injected with 4 mg/kg phencyclidine and placed in the Laboras equipment for one hour and the behaviours quantified.

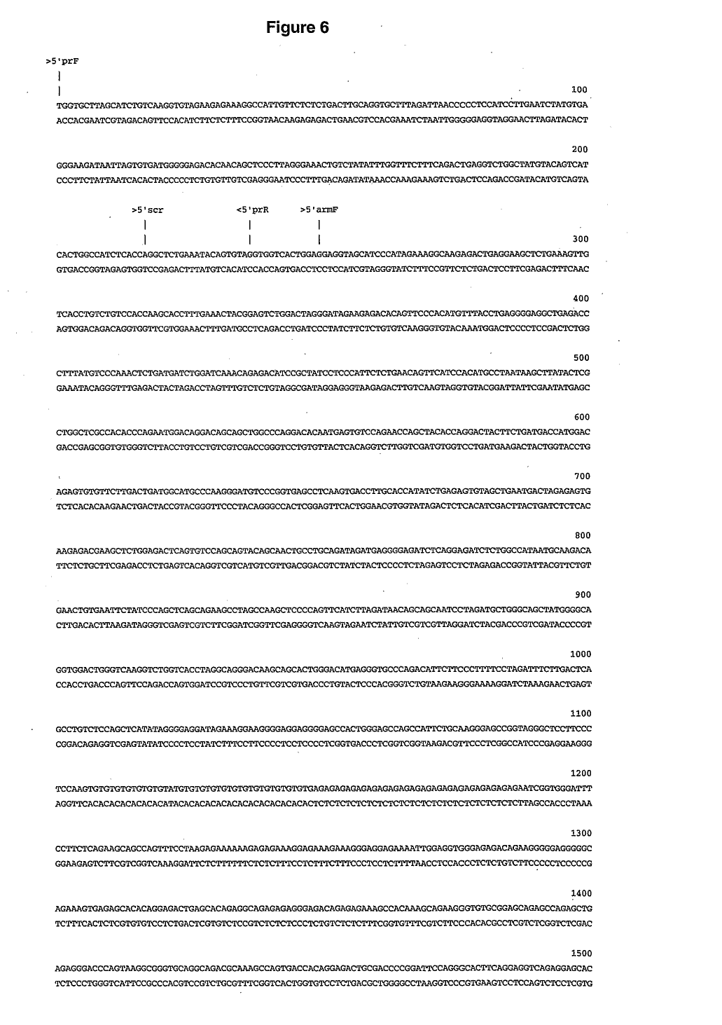

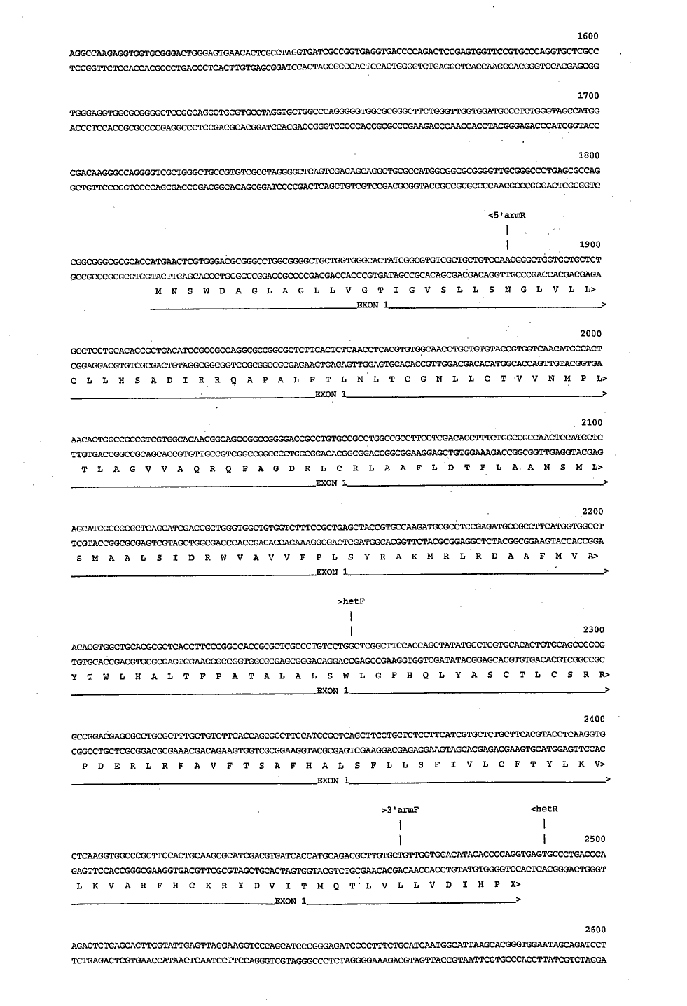

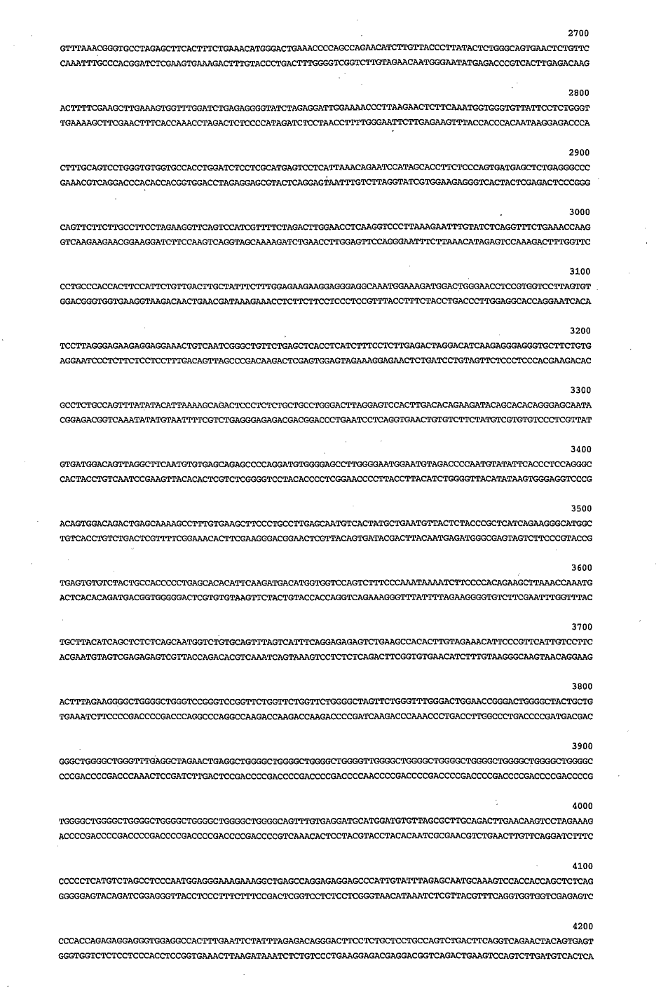

Phencyclidine in this study evoked a reduction in locomotor activity. This was interpreted to be the result of an increase in stereotypic behaviour. In knockout mice the response to phencyclidine was reversed, with the knockout mice showing a slight increase in locomotor activity following administration of phencyclidine presumably as a result of a reduction in stereotypic behaviour. The interaction between drug and genotype was statistically significant (P=<0.001). This data indicates that GPR26 knockout mice are resistant to the psychotomimetic effects of phencyclidine (see FIG. 5).

### Example 4

### Open Field

The open field comprises a large, square (60×60 cm) Perspex arena. The test is run under bright light. It measures a combination of locomotor activity, exploratory drive, neophobia, agoraphobia and other aspects of anxiety or fear in mice. In two separate open field tests it was noted that there was a trend for female KO mice to spend more time and rest longer in the central zone. This indicates that the mice are displaying ‘freezing’ behaviour, which indicates an anxious phenotype.

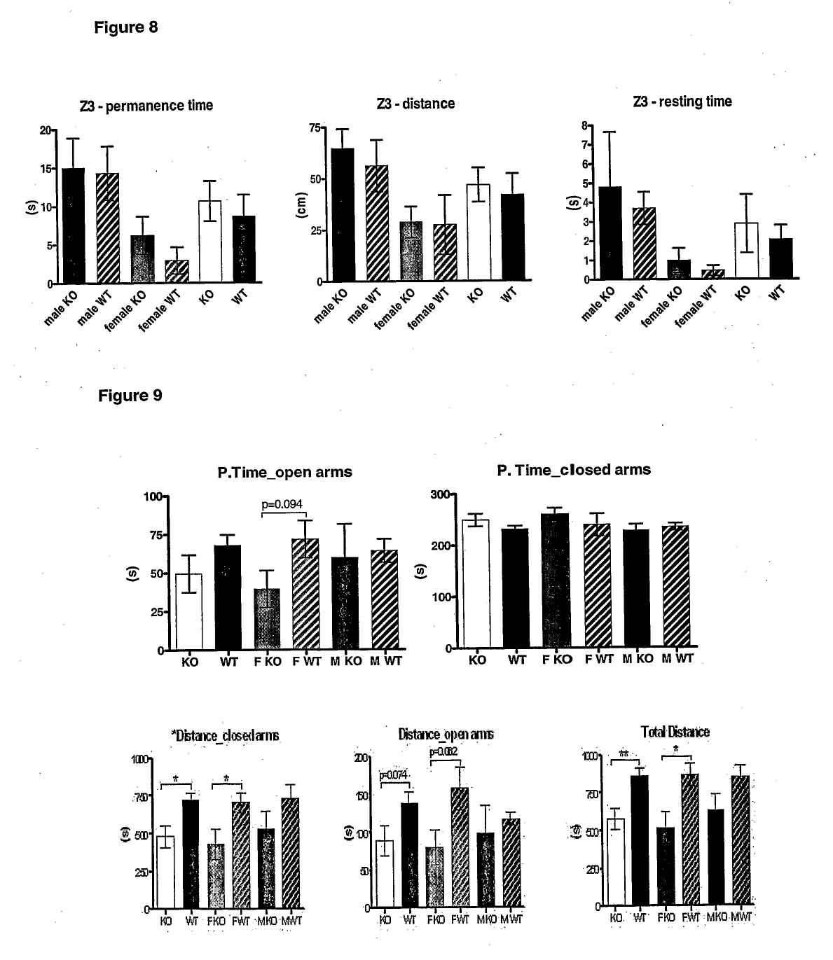

FIG. 8 is a graph showing the results of the open field for knockout (KO) compared to wildtype (WT) mice. (nb, Z3=central zone)

### Example 5

### Elevated Zero Maze

The zero maze consists of an annular platform (3 cm wide and 55 cm inside diameter) elevated 70 cm above the ground. It is divided in two opposite, enclosed quadrants (with black walls 21.5 cm high) and two open quadrants allowing uninterrupted exploration. The zero maze exploits the conflict between the tendency of mice to explore a novel environment and the aversive properties of a brightly lit open field, with the added components of height and openness. Female KO mice were noted to spend significantly less time in the open arms and more time resting in the closed arms. In addition, a significant reduction in distance traveled for KO mice was noted. This data indicates an increased anxiety phenotype in the KO mice. FIG. 9 is a graph showing the results of the elevated zero maze for knockout (KO) compared to wildtype (WT) mice. (nb, P.Time=time spent in arm)

### Example 6

### Elevated Plus Maze

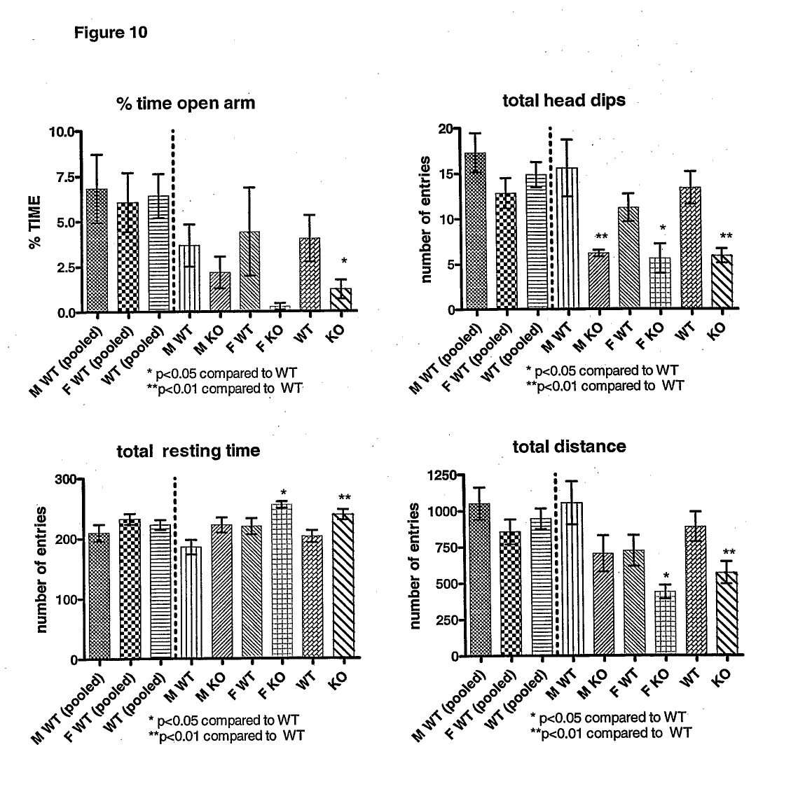

The plus maze consists of two open arms and two enclosed arms (with black walls 21.5 cm high), and one central platform (6×6 cm). The apparatus is elevated 70 cm above the ground. The plus maze exploits the conflict between the tendency of mice to explore a novel environment and the aversive properties of a brightly lit open field, with the added components of height and openness. KO mice were noted to spend significantly less time in the open arms and produce a significantly reduced number of head dips. KO mice were also observed to spend significantly more time resting. These data indicate a phenotype of increased anxiety in the KO mice. FIG. 10 is a graph showing the results of the elevated plus maze for knockout (KO) compared to wildtype (WT) mice.

Taken together, the data from the above tests (open field, elevated zero maze and elevated plus maze) indicate that GPR26 KO mice display an anxious phenotype. This would suggest that an agonist to GPR26 may exert an anxiolytic effect.

### Example 7

### MK-801-Induced Hyperlocomotion

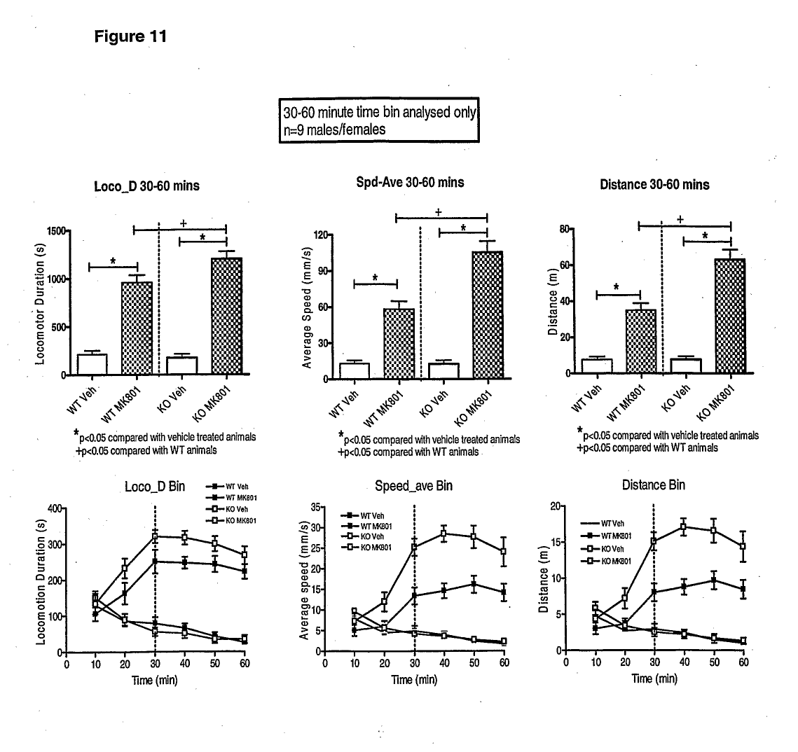

In this test mice are injected with MK-801 (0.3 mg/kg). MK-801 is a non-competitive NMDA channel blocker. It is known to increase locomotor activity at lower doses (at higher doses stereotypic behaviours are increased). The data from this test favours an increase in the locomotor response to NMDA channel blockade rather than a decrease in stereotypy (FIG. 11). This data indicates that in KO mice we observe an increase in the dopamine released by NMDA channel blockers in the nucleus accumbens. This data would translate to a psychogenic phenotype in these mice and suggest that an agonist at GPR26 may exert an antipsychotic effect. Alternatively, an antagonist may have utility in Parkinson's disease or ADHD. FIG. 11 is a graph showing the results of the MK-801-induced hyperlocomotion for knockout (KO) compared to wildtype (WT) mice.

### Example 8

### Watermaze

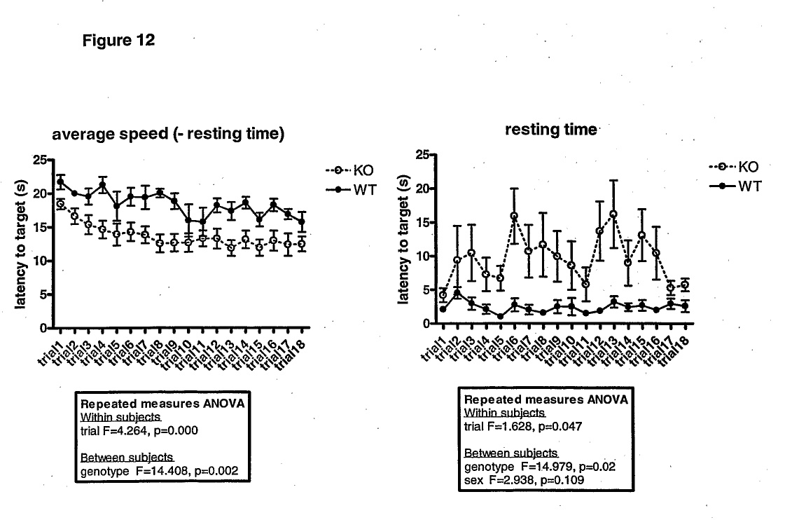

In this test mice are placed in a 1 m circular pool filled with water at 25±1° C. Mice are trained over 4 days to find a submerged platform. Average speed (average speed minus those periods spent resting) and time spent resting are significantly decreased and increased respectively in the KO group of animals. (FIG. 12) This would suggest that KO mice display a locomotor deficit.

Anxiety tests (elevated plus maze, elevated zero maze): Data obtained in these tests relating to time spent resting and distance traveled (increased and decreased respectively in the KO group) could indicate that KO mice display a locomotor deficit (FIGS. 9 and 10).

These data taken together indicate that an agonist acting at GPR26 could have a utility in a range of movement disorders e.g. Parkinson's disease, Huntington's disease.

### Example 9

### Behaviour: Pre Pulse Inhibition

Pre pulse inhibition (PPI) is an observed phenomenon whereby the startle response evoked by a loud (115 dB) stimulus is inhibited by a brief much quieter sound stimulus (3-10 dB above background) presented 100 ms before the startle stimulus. This so-called pre pulse inhibition is disrupted in schizophrenic patients and may reflect a deficit in psychomotor gating.

PPI can be evoked in rodents and disrupted by psychogenic and psychotomimetic drugs such as amphetamine, apomorphine, phencyclidine and MK801. This disruption can be reversed by antipsychotic drugs such as haloperidol, clozapine and olanzapine. (Bakshi V P, Swerdlow N R and Geyer M A. Clozapine antagonizes phencyclidine-induced deficits in sensorimotor gating of the startle response. J. Pharmacol. Exp. Ther. 1994; 271:787-794.) Therefore an animal model of PPI exists with both construct and predictive validity. Any effect of a drug or a mutation on PPI is a strong indication of potential utility in the therapy of schizophrenia. (McGhie A and Chapman J. Disorders of attention and perception in early schizophrenia. Br. J. Med. Psychol. 1961; 34:103-116. Braff D L, Geyer M A and Swerdlow N R. Human studies of prepulse inhibition of startle: normal subjects, patient groups, and pharmacological studies. Psychopharm. 2001; 156:234-258)

Animals are housed with free access to food and water under a light-dark cycle of 12 h light/12 h darkness with lights on at 7 am and are tested at set times to avoid circadian effects. Mice (6 KO mice of each sex with corresponding age matched wild type controls) are placed in a startle chamber (San Diego Instruments, San Diego Calif., USA). Each system consists of a sound attenuated chamber ventilated with a small fan and illuminated by a light bulb mounted in the ceiling. The chamber contains a transparent acrylic holding cylinder mounted to a frame. The cylinder and the frame are elevated about a Plexiglas base by four screws stationed under each corner of the frame. Acoustic stimuli are delivered through a small speaker mounted in the enclosure ceiling. Startle responses are transduced by the piezoelectric accelerometer mounted beneath the frame. Output signals are digitized, rectified and recorded as consecutive 1 millisecond (ms) readings on a PC with San Diego Instruments Startle Reflex software. The startle stimulus is a 115 dB pulse of broad spectrum noise 50 ms in length. The pre-pulse stimuli were 3, 7 and 10 dB above background and were presented 100 ms before the startle stimulus. Stimuli were presented in a pseudo-randomised fashion.

Animals are repeat tested for a PPI deficit in the presence and absence of a dopamine agonist; amphetamine, to assess any changes within this neurotransmitter system.

A separate squad of animals are repeat tested for a PPI deficit in the presence and absence of a non-selective NMDA receptor antagonist; dizocilpine (MK801), to assess other neurotransmitter changes. Antagonism, against the disruptive effect on PPI by these drugs in rodents has often been taken as evidence of potential antipsychotic properties (Geyer M A, Mcilwain K L and Paylor R. Mouse genetic models for prepulse inhibition: an early review. Mol. Psych. 2002; 7:1039-1053).

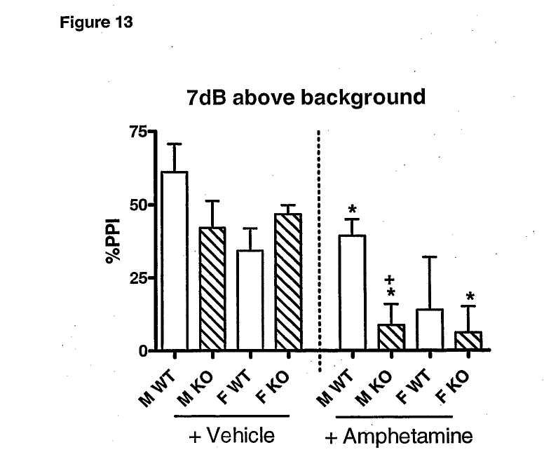

The results are shown in FIG. 13. KO animals not treated with amphetamine do not show any changes in PPI response in comparison to WT animals. The deficit caused to the PPI response by amphetamine was exacerbated in male KO animals in comparison with amphetamine-treated WT animals. This effect was most apparent at 7 dB above background, although the genotype effect was also seen at 10 dB above background.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of identifying an agent suitable for therapy of a mental illness comprising the step of determining whether a candidate agent affects the activity of GPR26.

2. The method according to claim 1, wherein the agent is an antagonist that inhibits the activity of GPR26.

3. The method according to claim 1, wherein the agent is an agonist that enhances the activity of GPR26.

4. The method according to claim 1, wherein the GPR26 comprises or encodes a polypeptide identified herein as SEQ ID No. 3 or SEQ ID No 5, or a sequence with at least 70% sequence identity thereto.

5. The method according to claim 1, wherein the GPR26 consists of or encodes a polypeptide identified herein as SEQ ID No 3, SEQ ID No 5, or a sequence with at least 70% identity thereto.

6. The method according to claim 1, wherein the mental illness is an anxiety disorder, a disease involving psychosis or a depressive disorder.

7. The method according to claim 1, wherein GPR26 polypeptide is contacted with the candidate agent to determine whether the candidate affects the activity of the GPR26.

8. The method according to claim 1, wherein the candidate agent is contacted with a cell expressing a GPR26 polypeptide.

9. The method according to claim 1, comprising the steps of administering the candidate agent to a non-human animal expressing a GPR26 polypeptide and determining whether the animal exhibits altered behaviour.

10. The method according to claim 9, wherein the non-human animal expresses functional GPR26 polypeptide.

11. The method according to claim 9, wherein the non-human animal is wild-type.

12. The method according to claim 9, wherein the non-human animal is a rodent.

13. A non-human transgenic animal having a functionality-disrupted endogenous GPR26 gene.

14. The non-human transgenic animal according to claim 13, wherein the animal has a null mutation.

15. The non-human transgenic animal according to claim 13, wherein the animal is −/− for the GPR26 gene.

16. A method for identifying an agonist or antagonist of GPR26 gene for the therapy of mental illness wherein said method comprises the use of a non-human transgenic animal having a functionality-disrupted endogenous GPR26 gene or an isolated cell or tissue thereof.

17. Use of a non-human transgenic animal having a functionality-disrupted endogenous GPR26 gene as a model for mental illness.

18. A method of determining the presence of, or susceptibility to, mental illness in an individual, comprising the steps of (i) determining the expression level of GPR26 gene in a sample isolated from the patient, and (ii) determining, compared to a control, whether the individual from which the sample was isolated has or is susceptible to mental illness.

19. A method for the prevention or treatment of mental illness wherein said method comprises administering, to a patient in need of such prevention or treatment,
a) an agent that affects the activity of a GPR26 polypeptide;
b) an exogenous GPR26 polynucleotide, or
c) an agent that increases or decreases the transcription or expression of a GPR26 polynucleotide.

20-21. (canceled)

22. The use according to claim 17, wherein the mental illness is an anxiety disorder, a disease involving psychosis or a depressive disorder.

23. The method, according to claim 18, wherein the mental illness is an anxiety disorder, a disease involving psychosis or a depressive disorder.

24. The method, according to claim 19, wherein the mental illness is an anxiety disorder, a disease involving psychosis or a depressive disorder.
